(19) United States
(12) Patent Application Publication
(10) Pub. No.: US 2004/0072777 A1
(43) Pub. Date: Apr. 15, 2004

(54) Epididymal antimicrobial peptides

(76) Inventors: Otto Froelich, Decatur, GA (US); Leona G. Young, Gainesville, GA (US)

Correspondence Address: JOHN S. PRATT, ESQ, KILPATRICK STOCKTON, LLP, 1100 PEACHTREE STREET, SUITE 2800, ATLANTA, GA, 30309, US

(21) Appl. No.: 10/381,752

(22) PCT Filed: Jan. 5, 2001
(86) PCT No.: PCT/US01/00432

(51) Int. Cl. A61K 48/00

(52) U.S. Cl. 514/44

(57) ABSTRACT

The present invention provides novel antimicrobial peptides expressed in the primate epididyrnis (hereinafter, “EP2 peptides”) and the nucleic acids encoding therefore. EP2 peptides and the nucleic acids encoding therefore can be administered to an individual having a microbial infection in an amount effective to treat the microbial infection or the endogenous production of EP2 peptides can be upregulated to an amount effective to treat the microbial infection. EP2 peptides are useful as antimicrobial agents in animals, including humans, and as antimicrobial agents in agricultural and industrial applications.

This invention was made in part with United States government support under National Institutes of Health Grant No. RR05994. The United States government has certain rights in this invention. This application claims priority of U.S. Application Serial No. 60/174,513, filed Jan. 5, 2000.

### FIELD OF THE INVENTION

The invention relates to novel epididymal antimicrobial peptides, and their use for the treatment of microbial infections.

### BACKGROUND OF THE INVENTION

Epithelia provide physical protection and antimicrobial peptides, synthesized by epithelia, provide chemical protection against potentially harmful agents in the environment (Maloy et al. Biopolymers 35:105, 1995).

These antimicrobial peptides are cationic and interact with the membrane of invading pathogens such as bacteria, fungi, viruses and parasites to cause disruptive changes in their permeability (Maloy et al. Biopolymers 37:105, 1995). Among these antimicrobial peptides are the defensins. Thus far, mammalian defensins are divided into α-defensins and β-defensins based on differences in the cross linking pattern of the six cysteine residues that stabilize their tertiary structures (White et al. Current Opinion in Structural Biology 5:521, 1995; Kagan et al. Toxicology 87:131, 1994). Mature α-defensins contain 29-35 amino acids and have a pair of cysteine residues (C₁ and C₂) near the N-terminus that are separated by one residue. Mature β-defensins contain 38-42 amino acids and have a pair of cysteine residues (C₁ and C₂) near the N terminus that are separated by six residues (White et al. Current Opinion in Structural Biology 5:521, 1995). Structurally, both α-defensins and β-defensins contain a hydrogen-bonded pair of antiparallel β strands connected by a short turn to form a β hairpin comprising the last 15 or so residues of the sequence.

In mammals, α-defensins have, thus far, been identified in lung macrophages, neutrophils, intestinal paneth cells and female reproductive tract and β-defensins have, thus far, been identified in neutrophils, in trachea, tongue, small intestine and female reproductive tract. In humans, α-defensins have, thus far, been identified in neutrophils, myeloid cells and paneth cells and β-defensins have, thus far, been identified in skin, tongue, salivary glands, prostate, trachea, lung, kidney and female reproductive tract (Valore et al. J. Clin. Invest 101:1633, 1998; Hiratsuka et al. Biochem. Biophys. Res. Commun. 249:943, 1998)

The mammalian epididymis is an epithelium that synthesizes peptides and secretes them into the lumen (Blaquier et al. Ann N.Y. Acad. Sci. 541:292, 1988; Hinton et al. Micros. Res. Tech. 30, 1995). Four epididymis-specific genes, HE1- HE4, were isolated from human epididymal cDNA library by differential screening for clones present in the epididymis but not testis (Kirchhoff et al. Int. J. Androl. 13:155, 1990). The nucleic acid sequence of the epididymis-specific gene HE2 corresponds to the nucleic acid sequence reported for EP2A (SEQ ID NO:32) (Fröhlich et al. J. Androl. 21:421, 2000). However, suggested uses for HE2 were limited to its possible use in the diagnosis of male infertility. In fact, prior to Applicants' invention, there had been no suggestion that antimicrobial peptides are synthesized and secreted by the epididymal epithelium.

Epididymitis, inflammation of the epididymis, is among the most common of human male complaints and also is a serious problem in the animal population. Causes of epididymitis include retrograde ascent of pathogens from the urogenital tract and spread of systemic infections to the epididymides. Pathogens that cause epididymitis include bacteria, fungi, viruses and parasites. Complications of epididymitis include, but are not limited to, testicular infarction, scrotal abscess, chronic-draining scrotal sinus and infertility. Moreover, epididymitis is an important focus of organisms causing bacteremia and local morbidity in patients with indwelling transurethral catheters.

Traditional treatment for epididymitis is the administration of antibiotics. However, as the emergence of antibiotic resistant strains of microbes has become more frequent, antibiotic administration has become less effective. Moreover, patient compliance with antibiotic regimens is frequently not well observed.

Therefore, there is a continuing need for novel antimicrobials that are effective against bacterial, fungi, viruses and parasites.

### SUMMARY OF THE INVENTION

The present invention addresses this need by providing an isolated nucleic acid having any one of the sequences corresponding to SEQ ID Nos:34-44, 49,-51, 54, 56, 58-62, 68 and 69, or degenerate variants thereof. The present invention also provides a novel antimicrobial peptide having any one of the sequences corresponding SEQ ID NOs:1-12 or fragments thereof. These peptides (hereinafter, “EP2 peptides”) can be administered to an individual having a microbial infection in an amount effective to treat the microbial infection or the endogenous production of EP2 peptides can be upregulated to an amount effective to treat the microbial infection. EP2 peptides are useful as antimicrobial agents in animals, including humans, and as antimicrobial agents in agricultural and in industrial applications.

Accordingly, it is an object of the present invention to provide EP2 peptides of mammalian epididymal origin.

It is another object of the present invention to upregulate expression of an EP2 peptide in mammalian epididymis.

It is another object of the present invention to provide EP2 peptides of primate epididymal origin.

It is another object of the present invention to upregulate expression of an EP2 peptide in primate epididymis.

It is another object of the present invention to provide a prepro-peptide precursor of an EP2 peptide.

It is another object of the present invention to provide a pro-peptide precursor of an EP2 peptide.

It is another object of the present invention to provide cDNA encoding an EP2 peptide.

It is another object of the present invention to provide cDNA encoding a prepro-peptide precursor of an EP2 peptide.

It is another object of the present invention to provide cDNA encoding a pro-peptide precursor of an EP2 peptide.

It is another object of the present invention to provide cDNA encoding a promoter-regulatory sequence positioned at the 5′-end of cDNA encoding an EP2 peptide.

It is another object of the present invention provide cDNA encoding an EP2 promoter-regulatory sequence positioned at the 5′-end of cDNA encoding an EP2 peptide.

It is another object of the present invention to provide a vector containing cDNA encoding an EP2 peptide.

It is another object of the present invention to provide a vector containing cDNA with an EP2 promoter-regulatory sequence positioned at the 5′-end of cDNA.

It is another object of the present invention to provide anti-sense DNA that is identical to the non-coding strand of the double stranded DNA encoding the EP2 gene.

It is another object of the present invention to provide anti-sense RNA that corresponds to the noncoding strand of the double-stranded DNA encoding the EP2 gene.

It is another object of the present invention to provide cells transfected with expression vectors for expressing EP2 peptides.

It is another object of the present invention to provide a composition and method for preventing a microbial infection.

It is another object of the present invention to provide a composition and method for preventing a microbial infection in an animal, including a human, by administering an EP2 peptide to the animal, including the human.

It is another object of the present invention to provide a method for preventing a microbial infection in an animal, including a human, by upregulating EP2 peptide expression in the animal, including the human.

It is another object of the present invention to provide a composition and method for preventing an infection in an animal, including a human, by inducing EP2 peptide expression, either intrinsic or extrinsic, in the animal, including the human

It is another object of the present invention to provide a composition and method for treating a microbial infection.

It is another object of the present invention to provide a composition and method for treating a microbial infection in an animal, including a human, by administering an EP2 peptide to the animal, including the human.

It is another object of the present invention to provide a method for treating a microbial infection in an animal, including a human, by upregulating EP2 peptide expression in the animal, including the human.

It is another object of the present invention to provide a composition and method for treating an infection in an animal, including a human, by inducing EP2 peptide expression, either intrinsic or extrinsic, in the animal, including the human.

It is another object of the present invention to provide an antimicrobial peptide useful in human medicine.

It is another object of the present invention to provide an antimicrobial peptide useful in veterinary medicine.

It is another object of the present invention to provide an antimicrobial peptide useful in agricultural science.

It is another object of the present invention to provide an antimicrobial peptide useful in industrial science.

It is another object of the present invention to provide an antimicrobial peptide effective against bacteria.

It is another object of the present invention to provide an antimicrobial peptide effective against fungi.

It is another object of the present invention to provide an antimicrobial peptide effective against viruses.

It is another object of the present invention to provide an antimicrobial peptide effective against parasites.

It is another object of the present invention to provide a panel of polyclonal antibodies and fragments thereof, each of which has the ability to bind to an EP2 peptide.

It is another object of the present invention to provide a panel of monoclonal antibodies and fragments thereof, each of which has the ability to bind to an EP2 peptide.

These and other objects, features and advantages of the present invention will become apparent after a review of the following detailed description of the disclosed embodiments and the appended claims.

### BRIEF DESCRIPTION OF THE FIGURES

FIG. 1. Graphic representation of the location of human and chimpanzee EP2 modules (SEQ ID NOs:25-31) within the human and chimpanzee EP2 peptides (SEQ ID NOs:1-12).

FIG. 2. Graphic representation of human and chimpanzee EP2 variant cDNAs (SEQ ID NOs:32-44) and EP2 peptides (SEQ ID NOs:1-12). The boxed regions delineate the open reading frames. The letters A and B indicate the two different leader sequences that are removed post-translationally at the signal cleavage sites (vertical arrows).

FIG. 3. Alignment of the amino acid sequences of human and chimpanzee module 3 (SEQ ID NOs:28&29) and module 4 (SEQ ID NOs:30 & 31) with the sequence of human mature β-defensin-1 (SEQ ID NO:63) DEFB1; Genbank accession number AAC51728) (Liu et al. Genomics 43:316-320 1997) and human mature β-defensin-2 (SEQ ID NO:64) (DEFB2; Genbank accession number AF071216) (Diamond et al. Infect. Immun. 68:113, 2000). The six cysteine residues (underlined) are the signature of β-defensins.

### DETAILED DESCRIPTION OF THE INVENTION

The present invention provides an isolated nucleic acid having any one of the sequences corresponding to SEQ ID Nos:34-44, 49,-51, 54, 56, 58-62, 68 and 69, and degenerate variants thereof. The present invention also provides a novel antimicrobial peptide having any one of the sequences corresponding SEQ ID NOs:1-12 or fragments thereof. These peptides (hereinafter, “EP2 peptides”) can be administered to an individual having a microbial infection in an amount effective to treat the microbial infection or the endogenous production of EP2 peptides can be upregulated to an amount effective to treat the microbial infection. EP2 peptides are useful as antimicrobial agents in animals, including humans, and as antimicrobial agents in agricultural and in industrial applications.

As used herein, the term “EP2 peptide” refers to the naturally occurring full length EP2 peptide as defined by the open reading frame, to synthetic or recombinant EP2 peptide, to fragments, derivatives and analogs thereof and to substitutions therein.

As used herein, the term “mature EP2 peptide” refers to the EP2 peptide after cleavage of the leader sequence, to synthetic or recombinant mature EP2 peptide, to fragments, derivatives and analogs thereof and to substitutions therein, wherein the mature EP2 peptide retains at least 25% of its activity as measured by minimal growth inhibitory concentration to Pseudomonas aeruginosa.

As used herein, the term “module” refers to a naturally occurring, synthetic or recombinant peptide sequence, to fragments, derivatives and analogs thereof and to substitutions therein, wherein one or more modules comprise an EP2 peptide.

As used herein, the term “nucleic acid” refers to a single stranded DNA sequence and a double stranded DNA sequence.

As used herein, the term “isolated nucleic acid” refers to a nucleic acid that is a cDNA, a genomic fragment, a fragment produced by polymerase chain reaction (PCR), a synthetic sequence, and a recombinant nucleic acid sequence that is part of a hybrid gene encoding a fusion protein.

As used herein, the terms “isolated peptide” or “isolated protein” refer to a peptide substantially free from other components in its in vivo cellular environment and, therefore, useful in ways that the non-isolated peptide is not useful.

As used herein, the terms “variant” or “degenerate variant” refer to any DNA sequence that codes for a corresponding EP2 peptide, mature EP2 peptide, EP2 module. EP2 fragment and modified EP2 peptide.

As used herein, the term “upregulation” refers to induction of endogenous EP2 expression and supplementation of EP2 expression by exogenous DNA.

As used herein, the term “microbe” refers to a bacterium, fungus, virus and parasite.

As used herein, the term “antibody” refers to any class of antibody and includes polyclonal antibodies and fragments thereof, monoclonal antibodies and fragments thereof, single chain recombinant antibodies and “humanized” chimeric antibodies.

As used herein, the term “pharmaceutical agent” includes any agent approved by a regulatory agency of a country or a state government or listed in the U.S. Pharmacopoeia (USP) or other generally recognized pharmacopoeia for use in an animal, including a human and any natural or non-synthetic agent that provides health benefits to an individual to whom the agent is administered.

The present invention relate to all mammalian epididymal EP2 peptides including, but not limited to, human and chimpanzee EP2A-EP2F (SEQ ID NOs:1-12) Each of these peptides has a consensus leader sequence typical for a secreted peptide. After removal of the leader sequence, human and chimpanzee mature EP2 peptides A-F (SEQ ID NOs:13-24) can be viewed as being comprised of one or more peptide modules selected from the group consisting of human and chimpanzee EP2 modules 1-4 (SEQ ID NOs:25-31).

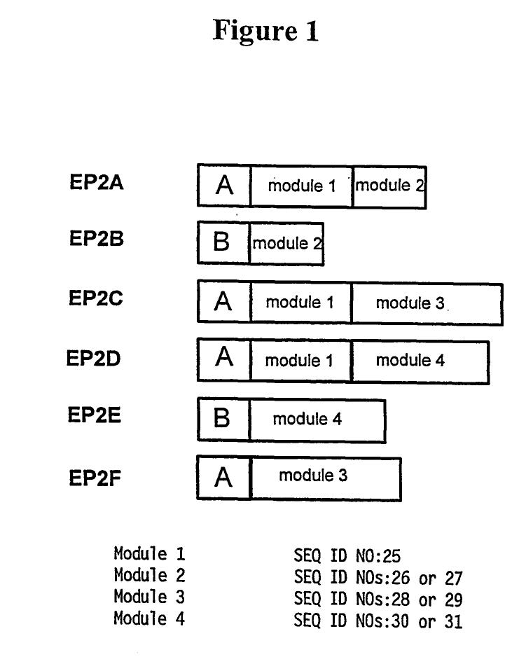

Human and chimpanzee EP2A peptide (SEQ ID NOs:1,2) is comprised of EP2 modules 1 (SEQ ID NO:25) and 2 (SEQ ID NOs:26,27). EP2B peptide (SEQ ID NOs:3,4) is comprised of EP2 module 2 (SEQ ID NOs:26,27). EP2C peptide (SEQ ID NOs:5,6) is comprised of EP2 modules 1 (SEQ ID NO:25) and 3 (SEQ ID NOs:28,29). EP2C=SEQ ID NOs:25+28 & 25+29. EP2D peptide (SEQ ID NOs:7,8) is comprised of EP2 modules 1 (SEQ ID NO:25) and 4 (SEQ ID NOs:30,31). EP2D=SEQ ID NOs:25+30 & 25+31. EP2E peptide (SEQ ID NOs:9,10) is comprised of EP2 module 4 (SEQ ID NOs:30,31). EP2F peptide (SEQ ID NOs:11,12) is comprised of EP2 module 3(SEQ ID NOs:28,29) (FIG. 1).

Although not wishing to be bound by the following hypothesis, it is believed that the human and chimpanzee EP2 peptides modules (SEQ ID NOs:13-24) relate to the maturation state of the EP2 peptides (SEQ ID NOs:1-12) and to their antimicrobial activity. Removal of the leader sequence from the full-length peptides EP2A (SEQ ID NOs:1,2), EP2C (SEQ ID NOs:5,6) and EP2D (SEQ ID NOs:7,8) results in the secreted peptides (SEQ ID NOs:13,14,17,18,19&20). These peptides contain module 1 (SEQ ID NO:25) as a prosequence whose enzymatic removal, either before or after secretion, turns them into biologically active module 2 (SEQ ID NOs:4,15), module 3 (SEQ ID NOs:16,17) or module 4 (SEQ ID NOs:18,19). As EP2 peptides EP2B (SEQ ID NOs:3,4), EP2E (SEQ ID NOs:8,9) and EP2F (SEQ ID NOs:10,11) do not contain module 1 (SEQ ID NO:25), removal of the leader sequence results directly in the biologically active peptides.

EP2 peptides include an EP2 peptide modified by the addition or removal of one or more amino acids from either or both ends of the peptide or from an internal region of the peptide, without substantial loss of its activity. For example, a tyrosine, labeled with a radioisotope or a lysine labeled with a chemical can be added to the first position of an EP2 peptide for use as a marker in diagnostic assays and to enhance the ability of the EP2 peptide to destroy a target, which contains EP2 peptide receptors. Further, EP2 peptides can be modified by a conservative substitution of one or more amino acids or by a non-conservative substitution of one or more natural or synthetic amino acids to increase or to decrease the bioactivity of the peptide or to produce biological or pharmacological agonists or antagonists of the peptide. EP2 peptides also include an EP2 peptide modified by derivatization of a peptide, glycosylation, deglycosylation and phosporylation.

The present invention also includes the human and chimpanzee nucleic acid variants (SEQ ID NOs:32-43) that encode the EP2 peptides (SEQ ID NOs:1-12), mature EP2 peptides (SEQ ID NOs:13-24) and EP2 modules (SEQ ID NOs:25-31), vectors containing these variants and cells and tissues transfected with these vectors that produce EP2 peptides. Further the present invention includes human and chimpanzee nucleic acid sequences that code for proteins comprising at least 20 contiguous residues of an amino acid sequence selected from the group consisting of SEQ ID NOs:28-31.

The nucleic acids having SEQ ID NOs:33, 35, 37 were obtained by phage plaque hybridization screening and by hybridization absorption. The nucleic acids having SEQ ID NOs:39, 41 and 43 were derived by aligning sequences obtained by PCR Fröhlich et al. J. Androl. 21:421, 2000). SEQ ID NO:44 was obtained by sequencing a genomic. clone. SEQ ID NOs:32, 34, 36, 38, 40 and 42 were derived from the SEQ ID NO:44 by alignment with the homologous chimpanzee SEQ ID NOs:33, 35, 37, 39, 41 and 43.

The present invention is further directed to fragments or variants of isolated nucleic acid, wherein the fragments or variants comprise contiguous bases of preferably about 10 to 100 nt, more preferably about 15 to 75 nt and most preferably about 20 to 40 nt contiguous nucleic acids derived from SEQ ID NO:44. These fragments or variants can be used as diagnostic probes, primers and hybridization probes. These fragments or variants hybridize under highly stringent hybridization conditions to a sequence or to a inverse complement sequence of SEQ ID NOs:34-44, 49-51, 54, 56, 58-62, 68 and 69 and fragments and variants thereof. A highly stringent hybridization condition is an overnight incubation at 42° C. in a solution comprising 50% formamide, 5×SSC (750 mM NaCl, 75 mM trisodium citrate), 50 mM sodium phosphate (pH 7.6), 5×Denbardt's solution, 10% dextran sulfate, and 20 μg/ml denatured, sheared salmon sperm DNA, followed by washing the filters in 0.1×SSC at about 65° C.

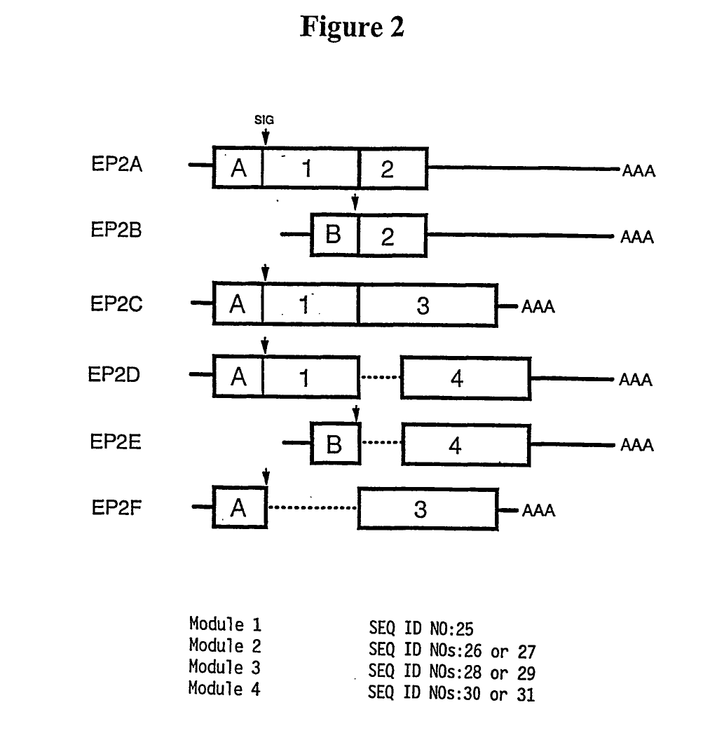

Although not wanting to be bound by the following hypothesis, it is thought that a single EP2 gene (SEQ ID NO:44) gives rise to the different EP2 variants (SEQ ID NOs:32-43) by transcription from two different promoters and by alternative splicing. The isolated EP2 variants differ in their 5′-end, in their 3′-end and in their inclusion or omission of an exon located in the open reading frame. The inclusion or omission of this exon results in a shift of the reading frame. This shift, in combination with the alternative use of different 5′- and 3′-cDNA ends, results in translation products some of which have no amino acid sequences in common with each other (FIG. 2).

An EP2 peptide can be prepared by methods well known in the art including, but not limited to, isolation from semen, manual polypeptide synthesis, automatic polypeptide synthesis (“Solid Phase Peptide Synthesis: A Practical Approach” Atherton et al. Eds., IRL Press, Oxford England; 1988), recombinant methods (Current Protocols in Molecular Biology, Ausubel et al. Eds. John Wiley & Sons, Inc., New York, 1998, incorporated by reference herein), introduction of a transgene into an animal, culture of genetically altered cells and implantation of genetically altered cells into an animal. To minimize potential inactivation by proteases, an EP2 peptide can be synthesized from D-amino acids (Wade et al. Proc. Natl. Acad. Sci. 87:4761, 1990).

An EP2 peptide is prepared using recombinant methods by inserting the nucleic acid encoding the EP2 peptide into a vector including, but not limited to, a plasmid, a virus and a baculovirus, and recombinantly expressing the EP2 peptide in living cells including, but not limited to, bacterial, mammalian, insect and yeast cells. It will be appreciated that “EP2 peptide” also encompasses a recombinant fusion peptide that includes any combination of EP2 modules 14 (SEQ ID NOs:25-31) and fragments thereof.

The isolated EP2 peptides of the present invention are preferably about 75% to 99% pure, more preferably about 80% to 98%, pure, and most preferably about 90% to 97% pure as measured by band intensity on a silver stained gel or other methods known in the art.

Polyclonal and monoclonal antibodies and variants thereof specific for an EP2 peptide are generated by methods well known in the art (“Antibodies: A Laboratory Manual” Harlow et al. Eds., Cold Spring Harbor Laboratory, Cold Spring Harbor, N.Y., 1988, incorporated by reference herein). Antibody titer is determined by methods including, but not limited to, ELISAs, dot blots and density analysis. A monoclonal antibody that binds specifically to an EP2 peptide can be isolated and purified and its amino acid sequence determined by methods known in the art.

An anti-EP2 peptide antibody can be used in competitive and non-competitive immunoassays including, but not limited to, ELISAs, dot blots, sandwich immunoassays and radioimmunoassays (RIAs), to detect or to quantify the amount of the EP2 peptide in a biological sample. In particular, the antibody may be used to detect or to quantify an EP2 peptide in urine, in semen and in reproductive tissue. Results from these assays may be used to diagnose or to predict the occurrence or reoccurrence of a microbial infection and, in particular, a microbial infection of the urogenital tract. In an example, the amount of EP2 peptide in a semen sample can be measured in an ELISA assay or in a dipstick assay, in which the amount of EP2 peptide is compared with a known normal amount. An EP2 peptide level above normal indicates the individual has a reproductive tract infection. Both the ELISA assay and the dipstick assay can be provided in a kit for use by a health provider or by the affected individual.

An EP2 peptide also can be used to isolate an EP2 receptor that specifically binds the EP2 peptide. The isolated and purified receptor can be sequenced so that the gene or genes coding for the receptor can be identified and sequenced.

Antibodies and receptors that bind an EP2 peptide with high specificity and avidity can be labeled with a reporter including, but not limited to, a fluorescent probe, a calorimetric probe, an isotope and an enzyme, and used to visualize the EP2 peptide in epididymal tissue and to quantitate the amount of the EP2 peptide in vivo and in vitro for diagnostic and research purposes.

Although EP2 peptide activity is not limited to antimicrobial activity, preferably an EP2 peptide has antimicrobial activity. This activity can be microbiostatic, wherein the EP2 peptide inhibits growth of a microbe, or microbiocidal, wherein the EP2 peptide kills or irreversibly damages a microbe. An EP2 peptide can be used in animals, including humans, as a microbiostatic to prevent a microbial infection or as a microbiocidal to treat a microbial infection. In an example, an EP2 peptide is used alone or in combination with a pharmaceutical agent to prevent the spread of a sexually transmitted disease by inclusion in condoms for use by both males and females, in spermicidal creams and jellies, in vaginal lubricants and in vaginal sponges. In another example, an EP2 peptide is used to treat a sexually transmitted disease by administration to an animal, including a human. In another example, an EP2 peptide is used to treat a sexually transmitted disease by upregulation of EP2 peptide expression in an animal, including a human. Sexually transmitted diseases include, but are not limited to, Neisseria gonorrhoeae, Chlamydia trachomatis, Pseudomonas aeruginosa, Escherichia coli and Candida albicans. In another example, an EP2 peptide is used to treat infection of the ear, eye, skin, epithelia and mucus membranes. In another example, an EP2 peptide can be used in agricultural and industrial applications as a microbiostatic to prevent microbial contamination and as a microbiocidal to eliminate microbial contamination. In another example, an EP2 peptide can be used in food products as food preservative and as a microbiostatic and a microbiocidal in disinfectants, shampoos, deodorants, soaps, detergents and cleaning products.

Microbial infections of the urogenital tract are caused by bacteria, fungi, viruses and parasites. Bacteria include, but are not limited to, Neisseria gonorrhoeae, Chlamydia trachomatis, Pseudomonas aeruginosa, Escherichia coli, Staphylococcus aureus, Mycobacterium tuberculosis, Treponema pallidum, Trichomonas vaginalis, Neisseria meningitidis, Haemophilus influenzae, Streptococcus pneumoniae, Brucella abortus and Brucella melitensis. Fungi include, but are not limited to Aspergillus fumigatus, Candida albicans and Candida tropicalis. Viruses include, but are not limited to Cytomegalovirus, ovine lentivirus (OvLv). Parasites include, but are not limited to, filaria, schistosoma and amebae.

Although different EP2 peptides will have different degrees of activity towards different microbes, the amount of an EP2 peptide effective to prevent or to treat a microbial infection can be determined readily by one skilled in the art. For example, a microbe can be grown to appropriate concentration, mixed with appropriate medium, plated and contacted with serial dilutions of an EP2 peptide. After appropriate incubation, the antimicrobial activity of the EP2 peptide is apparent from clear zones surrounding the EP2 sample. The clear zones are concentration dependent and, thus, enable the antimicrobial activity of the EP2 peptide against a given microbe to be determined.

For use, one or more EP2 peptides are formulated in a pharmaceutically acceptable carrier including, but not limited to, a liquid carrier, a solid carrier or both. Such compositions contain preferably from about 0.001 to 50% by weight, more preferably from about 0.01 to 20% and most preferably from about 0.1 to 10% of EP2 peptide.

Liquid carriers are aqueous carriers, non-aqueous carriers or both and include, but are not limited to, physiological buffers, oil emulsions, oil and water emulsions and liposomes. Solid carriers are biological carriers, chemical carriers or both and include, but are not limited to, viral vector systems, microparticles, nanoparticles, microspheres, nanospheres, minipumps, bacterial cell wall extracts and biodegradable or non-biodegradable natural or synthetic polymers that allow for sustained release of an EP2 peptide. Such polymers can be delivered into the vicinity of where delivery is required. Polymers and their use are described in, for example, Brem et al., J. Neurosurg. 74: 441-446 (1991). Methods used to complex EP2 peptides to a solid carrier include, but are not limited to, direct adsorption to the surface of the solid carrier, covalent coupling to the surface of the solid carrier, either directly or via a reversible or irreversible linking moiety, and covalent coupling to the polymer used to make a solid carrier.

Depending on the microbial infection to be prevented or treated, one or more pharmaceutical agents may optionally be included in an EP2 peptide formulation regardless of the pharmaceutically acceptable carrier used to administer the EP2 peptide. In addition, any one, all, or any combination of excipients may optionally be included in an EP2 peptide formulation. Such excipients include, but are not limited to, anti-oxidants, polyols, inert powders, suspending agents and thickening agents. It should be understood that, in addition to the ingredients particularly mentioned above, the formulations of the present invention can include other agents conventional in the art having regard to the type of formulation in question.

One or more EP2 peptides are administered to an animal having a microbial infection in an amount effective to treat the microbial infection. The amount of EP2 peptide administered per dose will depend on the EP2 peptide being used and the microbial infection being treated and preferably is about 0.001 to 5000 μg, more preferably about 0.01 to 2000 μg and most preferably from 0.1 to 500 μg. The particular EP2 peptide administered, the amount per dose, the dose schedule and the route of administration should be decided by the practitioner using methods known to those skilled in the art and will depend on the type of microbial infection, the severity of the microbial infection, the location of the microbial infection and other clinical factors such as the size, weight and physical condition of the recipient. In addition, in vitro assays may optionally be employed to help identify optimal ranges for EP2 peptide administration.

Routes of administration for EP2 peptides include, but are not limited to, oral, topical, transdermal, subdermal, subcutaneous intra-muscular, intra-peritoneal, intra-arterial, intra-venous, intra-dermal, intra-cranial, intra-lesional, intra-ocular, intra-pulmonary, intra-spinal, placement within cavities of the body, nasal inhalation, pulmonary inhalation, impression into skin and electroporation. Topical formulations include, but are not limited to a rinse, powder, cream, ointment, gel, suppository and spray. EP2 peptides also can be delivered by cannula to the site of interest and, for sustained delivery, by the use of osmotic mini-pumps. Further, EP2 peptides may be incorporated into or applied to the surface of devices including, but not limited to, implants, stents, catheters, surgical instruments, condoms, diaphragms and intra-uterine devices.

Depending on the route of administration, the volume per dose is preferably about 0.001 ml to about 100 ml per dose, more preferably about 0.01 ml to about 50 ml per dose and most preferably about 0.1 ml to about 30 ml per dose. The EP2 composition can be administered in a single dose treatment or in multiple dose treatments on a schedule and over a period of time appropriate to the half-life of the EP2 peptide used, the infection being treated, the condition of the recipient and the route of administration.

An isolated DNA encoding an EP2 peptide also can be used to treat epithelial infections in an animal, including a human. For example, naked DNA encoding an EP2 peptide can be administered to an animal, including a human, as naked DNA, as lipid or peptide encapsulated DNA, as vector incorporated DNA, wherein the DNA encoding the EP2 peptide expresses the EP2 peptide within the cells of the animal, including the human For example, a viral vector including, but not limited to, an adenovirus, an adeno-associated virus or a retrovirus containing DNA encoding an EP2 peptide can be administered into the epididymides of an animal, including a human, having epididymitis, wherein the EP2 peptide is expressed in the cells of the epididymides and is secreted into the lumen in an amount effective to treat the epididymitis.

This invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLE 1

Screening for EP2 cDNAs

A cDNA library was generated from a chimpanzee epididymis in lambda ZAP phage (Stratagene, LaJolla, Calif.). This cDNA library was screened by conventional plaque hybridization screening and by hybridization adsorption (Fröhlich et al. J. Androl. 21:421, 2000). For plaque hybridization screening, established protocols were followed (Current Protocols in Molecular Biology, Ausubel et al. Eds. John Wiley & Sons, Inc., New York, 1998). Briefly, bacteria were infected with the phage library and spread as a lawn. Phage plaques were adsorbed onto nitrocellulose filters and the filters were developed using a ³²P-labeled probe prepared from the subdloned EP2A PCR fragment (SEQ ID NO:45) using random hexamers and Klenow fragment. After two rounds of plaque purification, the plasmid cDNA was rescued using helper phage.

Hybridization adsorption was done using the GeneTrapper kit (Life Technologies, Gaithersbug, Md.) according to the accompanying protocol. Briefly, the phage library was converted into a plasmid library using helper phage and then converted from double-stranded to single-stranded cDNA using the Gene II peptide of bacteriophage f1 in combination with exonuclease III. A biotin-labeled reverse primer (EP2PCR4, 5′-GGGATCAGAGCAAATGTCACGC-3′, SEQ ID NO:46) was hybridized to the single-stranded cDNA library and reacted with matrix-bound streptavidin to bind all specifically hybridized plasmids to the matrix. After elution from thematrix, the single-stranded cDNA was converted back to double-stranded cDNA, the double stranded plasmid was transformed into bacteria and the resulting cDNA clones were sequenced.

Total RNA was isolated from epididymides, recovered surgically from adult male chimpanzees, and aliquots of the RNA were reverse-transcribed using the Superscript Preamplification System (Life Technologies, Gaithersbug, Md.). The resulting cDNA was used for PCR. PCR was performed with Taq DNA polymerase (Perkin-Elmer, Branchburg, N.J.; Life Technologies, Gaithersburg, Md.) and the following cycling protocol: 4 minutes at 92° C., followed by 25-30 cycles of 1 minute at 92° C., 1 minute at 58°, 60°or 62° C., 1-3 minutes at 72° C., followed by 10 minutes at 72° and at 4° C. until the samples were recovered from the cycler. PCR products were analyzed on 1.5% agarose gels or on 8% polyacrylamide gels in TAE buffer, using the 1 kb DNA ladder (Life Technologies, Gaithersburg, Md.) as a standard. For sequence identification, the PCR product bands were subcloned into a TA vector (pGEM-T Easy, Promega Life Sciences, Madison, Wis.) and sequenced using the Sequenase II system (Amersham Life Sciences, Cleveland Ohio) (Fröhlich et al. J. Androl. 21:421, 2000).

### EXAMPLE 2

Identification of Chimpanzee EP2A-EP2F Variants (SEQ ID NOs:33, 35, 37, 39, 41 and 43)

Chimpanzee EP2 variant EP2A (SEQ ID NO:33) was obtained by phage plaque hybridization. A 404 bp PCR product (SEQ ID NO:45), obtained by reverse-transcribing and amplifying (RT-PCR) epididymal RNA with the primers EP2PCR3, 5′-AGACATGAGGCAACGATTGCTCC-3′ (SEQ ID NO:47) and EP2PCR4 (SEQ ID NO:46), was used as probe. The open reading frame of variant EP2A (SEQ ID NO:48) is 309 bp in length and codes for the EP2A peptide (SEQ ID NO:2) of 103 amino acid residues, a molecular weight of 11.3 kDa and a pI of 11.5. The mature, secreted EP2A peptide (SEQ ID NO:14) contains 79 amino acid residues and has a glycosylation consensus sequence near the N-terminus. Without glycosylation, mature EP2A peptide (SEQ ID NO:14) has a molecular weight of 8.7 kDa and a pI of 10.8. Mature EP2A peptide (SEQ ID NO:14) is comprised of EP2 module 1 (SEQ ID NO:25) and EP2 module 2 (SEQ ID NO:27).

EP2 variant EP2B (SEQ ID NO:35) was obtained by hybridization adsorption. As probe to adsorb single-stranded cDNA plasmid clones containing EP2 inserts, biotinylated primer EP2PCR4 (SEQ ID NO:46) was used for adsorption, and unbiotinylated primer EP2PCR4 (SEQ ID NO:46) was used to prime the second-strand synthesis of the isolated plasmids. The open reading frame of variant EP2B (SEQ ID NO:49) is 150 bp in length and codes for EP2B peptide (SEQ ID NO:4) of 50 amino acid residues, a molecular weight of 5.6 kDa and a pI of 9.4. The mature, secreted, EP2B peptide (SEQ ID NO:16) contains 34 amino acid residues, has no glycosylation consensus sequence, a molecular weight of 3.7 kDa and a pI of 9.5. The mature EP2B peptide (SEQ ID NO:16) is comprised of EP2 module 2 (SEQ ID NO:27).

EP2 variant EP2C (SEQ ID NO:37) was obtained in the same phage plaque hybridization experiment as EP2A (SEQ ID NO:33). The open reading frame of variant EP2C (SEQ ID NO:50) is 339 bp in length and codes for EP2C peptide (SEQ ID NO:5), which is 113 residues in length, has a molecular weight of 12.7 kDa and a pI of 8.6. The mature, secreted, EP2C peptide (SEQ ID NO:18) contains 89 amino acid residues and has a glycosylation consensus sequence near the N-terminus. Without glycosylation, mature EP2C peptide (SEQ ID NO:18) has a molecular weight of 10.1 kDa and a pI of 8.1. The mature EP2C peptide (SEQ ID NO:18) is comprised of EP2 module 1 (SEQ ID NO:25) and EP2 module 3 (SEQ ID NO:29).

EP2 variant EP2D (SEQ ID NO:39) was identified by RT-PCR of epididymal RNA using the primers EP2PCR3 (SEQ ID NO:47) and EP2PCR4 (SEQ ID NO:46), as an electrophoretic band 76 bases smaller than the simultaneously obtained PCR product that is derived from EP2 variant EP2A (SEQ ID NO:33). The open reading frame of variant EP2D (SEQ ID NO:51) was obtained by RT-PCR of epididymal RNA using the primers EP2PCR3 (SEQ ID NO:47) and EP2STS2, 5′-CCCTTGGGATACTTCAACAT-3′ (SEQ ID NO:52). The open reading frame for variant EP2D (SEQ ID NO:51) is 399 bp in length and codes for EP2D peptide (SEQ ID NO:8) of 133 amino acid residues, which has a molecular weight of 14.9 kDa and a pI of 8.8. The secreted, mature EP2D peptide (SEQ ID NO:20) contains 109 amino acid residues and has a consensus glycosylation site near the N-terminus. Without gycosylation, mature EP2D peptide (SEQ ID NO:20) has a molecular weight of 12.3 kDa and a pI of 8.3. The mature EP2D peptide (SEQ ID NO:20) is comprised of EP2 module 1 (SEQ ID NO:25) and EP2 module 4 (SEQ ID NO:31).

EP2 variant EP2E (SEQ ID NO:41) was identified by RT-PCR of epididymal RNA using the primers EP2PCR5, 5′-GGCAGGGAGGTTCAACGGAC-3′ (SEQ ID NO:53) and EP2PCR4 (SEQ ID NO:46), as an electrophoretic band 76 bases smaller than the simultaneously obtained PCR product that is derived from EP2 variant EP2B (SEQ ID NO:33). The entire open reading frame of variant EP2E (SEQ ID NO:54) was obtained by RT-PCR of epididymal RNA using primers EP2PCR5 (SEQ ID NO:53) and EP2STS2 (SEQ ID NO:52). The open reading frame of variant EP2E (SEQ ID NO:54) is 240 bp in length and codes for EP2E peptide (SEQ ID NO:10) of 80 residues, which has a molecular weight of 9.1 kDa and a pI of 7.6. The mature, secreted EP2E peptide (SEQ ID NO:22) contains 64 amino acid residues, has a molecular weight of 7.2 kDa and a pI of 6.9. Mature EP2E peptide (SEQ ID NO:22) is comprised of EP2 module 4 (SEQ ID NO:31).

EP2 variant EP2F (SEQ ID NO:43) is obtained by RT-PCR of epididymal RNA using primers EP2PCR3 (SEQ ID NO:47) and EP2GEN9R, 5′-CATCAGTTTTAATGTAAACAGCAGGCGTC-3′ (SEQ ID NO:55), as an electrophoretic band 153 bases smaller than the simultaneously obtained PCR product that is derived from EP2 variant EP2B (SEQ ID NO:33). The open reading frame of variant EP2F (SEQ ID NO:56) is 186 bp in length and codes for EP2F peptide (SEQ ID NO:12) of 62 residues, a molecular weight of 7.1 kDa and a pI of 7.7. The secreted, mature EP2F peptide (SEQ ID NO:24) of 41 amino acid residues has no glycosylation consensus sequence and has a molecular weight of 4.8 kDa and a pI of 6.9. The mature EP2F peptide (SEQ ID NO:24) is comprised of EP2 module 3 (SEQ ID NO.:29).

### EXAMPLE 3

Homology of EP2 Peptides with Beta-Defensins

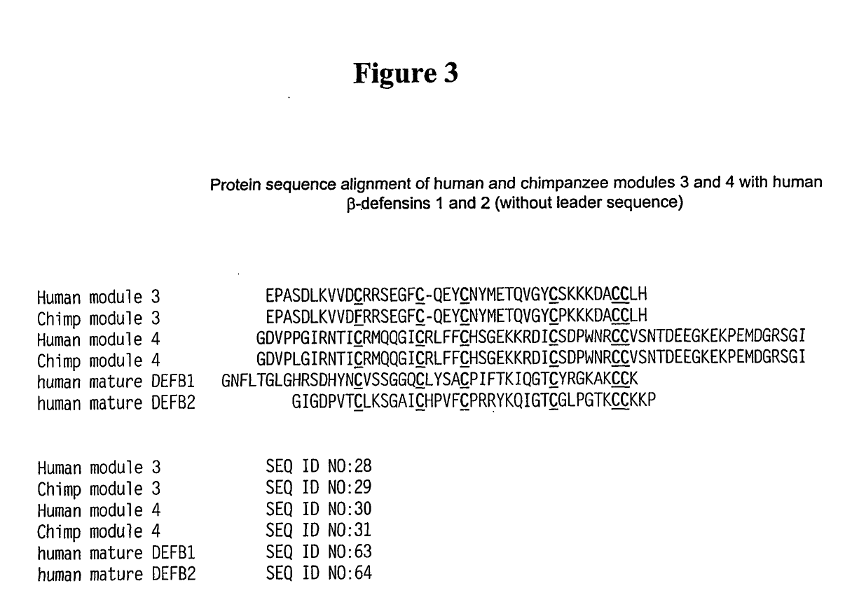

β-defensins are cationic peptides of 38-42 amino acids that contain six disulfide-linked cysteines. The 1st and 2nd cysteines are separated by six residues, the 2nd and 3rd by three or four residues, the 3rd and 4th by nine residues, the 4th and 5th by six residues, and the 5th and 6th are adjacent and the cysteines are disulfide bonded 1-5, 2-4, and 3-6 (Tang et al. J. Biol. Chem. 268:6649-6653, 1993). FIG. 3 shows that EP2 module 3 (SEQ ID NOs:28&29) and EP2 module 4 (SEQ ID NOs:30&31) show homology with β-defensins (SEQ ID NOs:63&64). However, the 1st cysteine in human EP2 module 3 (SEQ ID NO:28) is replaced by a phenylalanine in chimpanzee EP2 module 3 (SEQ ID NO:29).

### EXAMPLE 4

Localization of the EP2 Gene on Human Chromosome 8

A human genomic EP2 clone was custoisolated by Genome Systems (St. Louis, Mo.) from a PAC (P1 artificial chromosome) library using the STS primers EP2STS1 5′-GACATTTGCTCTGATCCCTG-3′ (SEQ ID NO:65) and EP2STS2 (SEQ ID NO:52). The insert of the resulting PAC clone (clone address: PAC-157(10E)) was estimated to be approximately 100-130 kb. The DNA sequence of the human EP2 gene (SEQ ID NO:44) was determined using PCR and sequencing to bridge presumed introns and primer walking to sequence into regions of unknown sequence. The program SeqMan of the Lasergene suite of cloning programs (Emory University Biomolecular Computing Resource) was used to combine all sequences into a single contiguous sequence. The human EP2 gene sequence (SEQ ID NO:44) is approximately 20 kb long and contains all exons that comprise the variants EP2A-EP2F (SEQ ID NOs:32, 34, 36, 38, 40&42). The overlapping chimpanzee cDNA and human genomic sequences are 99% identical.

The human EP2 peptide message (HE2) has been used as a STS marker for the human genome project (marker ID SHGC-11992 on Gene Map 98). This region of the chromosome contains all tested α- and β-defensins (Harder et al. Genomics 15:472-475, 1997; Linzmeier et al. Gene 233:205-11, 1999). Using yeast artificial chromosomes (YACs) mapped to this region, ⊖-defensin-1 (gene locus DefB1) and β-defensin-2 (gene locus DefB2) were located to the region between the anchor markers D8S550 and D8S552 defensins (Harder et al. Genomics 15:472-475, 1997). Using the same panel of YACs with the EP2-specific primers EP2PCR5. (SEQ ID NO:53) and EP2PCR4 (SEQ ID NO:46) and with the Defb2 primers DEFB2F 5′-GGCCCCAGTCACTCAGGAGAGATC-3′ (SEQ ID NO:66) and DEFB2R 5′-CGCATCAGCCACAGCAGCTTC-3′ (SEQ ID NO:67) as controls, EP2 was located in the vicinity of DefB2. Moreover, using PCR and the DefB2 primers DEFB2F (SEQ ID NO:66) and DEFB2R (SEQ ID NO:67), the DefB2 gene and the EP2 gene were found in the PAC genomic clone and are thus located within approximately 100 kb of each other. Combining this information with the genomic alignment of α- and β-defensin genes (Linzmeier et al. Gene 233:205-11, 1999) in the order of α- and β-defensins (from telomer to centromer) on human chromosome 8 is DefA5, DefA1/A, DefA1, DefA4, DefA6, DefB1, DefB2 and EP2.

The human EP2 gene has two promoters, promoter A (SEQ ID NO:68) and promoter B (SEQ ID NO:69) from which the different EP2 variant messages are transcribed. Promoter A drives the expression of variants EP2A, EP2C, EP2D and EP2F, while promoter B drives the expression of variants EP2B and EP2E. Both promoters contain consensus elements for binding of transcription factors that confer the epididymis-specific and hormone-dependent gene expression. Among others, promoter A contains seven hormone response element (HRE) half-sites, 5′-TGTTCT′-3, within the proximal 3 kb. HREs, which have the consensus sequence 5′-TGTTCTNNNAGAACA-3′, are the sequences on the promoter to which the group of nuclear receptors binds in their role of transcription factors, which includes the androgen receptor, the mineralocorticoid receptor, the glucocorticoid receptor and the progesterone receptor. One or several of these sites may therefore participate in the androgen dependence of EP2 expression. Promoter A also contains several sites for the transcription factor PEA3, which is expressed in the epididymis (Lan et al. Biol Reprod 60:664, 1999). Promoter B also contains several HRE half-sites and at least three sites .that have high homology to the full-length HRE. In addition, promoter B also contains several potential binding sites for PEA3.

### EXAMPLE 5

Induction of EP2 Peptides

Epididymal expression of EP2 peptides is regulated by androgens, which act at the level of nuclear DNA to regulate gene expression (Young et al. J. Reprod. Fertil. Suppl. 53:215, 1998). Hypogonadotrophic adult male chimpanzees were castrated unilaterally and the epididymides preserved for molecular-biological, studies. Using RT-PCR and Northern hybridization analysis, message for EP2 was detected in epididymides from androgen-normal chimpanzees and not in androgen-normal testis androgen-deprived epididymis and androgen-deprived testis (Young et al. J. Reprod. Fertil. Suppl. 53:215, 1998).

Other agents that may induce endogenous expression of EP2 peptides, include, but are not limited to, bacterial components including, but not limited to, lipopolysaccharides, glycolipids, glycopeptides and sugars, viral components, fungal components and parasitic components. Agents that induce endogenous expression of EP2 peptides can be determined using standard screening assays well known to those skilled in the art (Brey et al. Proc. Natl. Acad. Sci. USA 90:6275, 1993; Diamond et al. Chest 105S:51s, 1994).

### EXAMPLE 6

Synthesis of EP2 Peptides

EP2 peptides are synthesized in the Emory University Microchemical Facility by automated peptide synthesis using Applied Biosystems 430A(tBoc) and 433A(Fmoc) pepetide synthesizers and a Walters Delta Prep 3000 preparative HPLC by methods known to those skilled in the art. The purity of the synthesized peptides is assayed by mass spectroscopy using a PE-Sciex Model API3000 Triple Quadrupole Mass Spectrometer. Formation of the specific disulfide bonds in the EP2 peptides is achieved using methods known in the art (Kellenberger et al. Peptide Res. 8:321, 1995; Application Ser. No. PCT/US97/14639).

Unless stated otherwise, the. synthesized peptides are dissolved in buffer at 1-5 mg/ml and are stored at −20° C.

### EXAMPLE 7

Recombinant Peptides

EP2 peptides are expressed in cultured mammalian cells. The open reading frame of an EP2 variant is produced by PCR using primers that contain the open reading frame's 5′- or 3′-terminal nucleotides at their 3′-end and a restriction site for subcloning at their 5′-end, using methods known in the art. The PCR product is inserted into a vector that drives expression of the peptides off its promoter. The plasmid is introduced into human embryonic kidney (HEK293) cells by electroporation or using transfection agents and the cells are selected for stable transfectants. Stable transfectants are grown up clonally and selected for their levels of expression of EP2 peptide. To isolate the EP2 peptide, the cells are grown in the absence of antibiotics in defined medium. The EP2 peptide is isolated from the growth medium by ultrafiltration using a cutoff size of 20 kDa, followed by purification using chromatographic methods known to those skilled in the art.

Alternatively, EP2 peptides are expressed using the Drosophila expression system of Invitrogen (Carlsbad, Calif.). This system includes the Drosophila-derived Schneider S2 cell line, and a set of simple expression plasmid vectors for heterologous expression. The vectors contain either the metallothionein promoter for inducible expression or the Ac5 promoter for constitutive expression. Depending on which cloning sites on the vector are used, the expressed peptide can be fused to a V5 epitope tag for antibody recognition and a polyhistidine tag for affinity purification. The open reading frames are produced by PCR using existing cDNA or PCR clones as templates.

Alternatively, EP2 peptides are expressed using the Sf9/Baculovirus system (BD PharMingen, San Diego, Calif.). The vectors use the strong polyhedrin promoter in front of the polylinker. Depending on the vector, the mature form of the EP2 peptide is either expressed as fusion peptides with glutathione-S-transferase (GST), a His₆-tag and thrombin cleavage site for affinity purification and subsequent splitting of the fusion peptide into EP2 peptide and GST, or the cDNA encoding mature EP2 peptide is cloned. behind a baculovirus-encoded leader sequence for secretion of the mature EP2 peptide into the culture medium. The open reading frames are produced by PCR using existing cDNA or PCR clones as templates.

### EXAMPLE 8

Isolation of EP2 Peptides from Semen

Ejaculates are obtained from human donors. Sperm are separated from seminal fluid by centrifugation at 5000 g for 10 min at RT. The seminal fluid is neutralized to pH 7.0 with ammonium hydroxide. The neutralized seminal fluid is extracted with weak cationic exchange beads at 100 μl of a 50% slurry per 10 ml of seminal fluid by mixing for 2-4 h at RT, and then the beads are allowed to settle overnight at 4° C. The beads are washed with 25 mM ammonium acetate, pH 7.5, the peptides are batch eluted with 5% acetic acid and further purified by HPLC (Valore et al. J. Clin. Invest. 101:1633-1642, 1998).

The major HPLC peptide peaks are analyzed by acid-urea polyacrylamide gel electrophoresis and transferred to Immobilon-P polyvinylidene difluoride (PVDF) membranes (Millipore Corp., Bedford, Mass.). The membranes are stained with Cooomassie blue to identify peptides staining as single bands. For identification of the peptides, each band is cut from the membrane and NH₂-terminal amino acid sequenced. Further, the major HPLC peptide peaks are assayed for their antimicrobial activity on lawns of plated Neisseria gonorrhoeae, Chlamydia trachomatis, Pseudomonas aeruginosa and Escherichia coli.

### EXAMPLE 9

Generation of Polyclonal Antibodies

Mature EP2B peptide was synthesized and was conjugated to KLH at the Emory University Microchemical Facility (Atlanta, Ga.). Polyclonal antisera were raised in adult white New Zealand female rabbits to the KLH-conjugated EP2B peptide by AnaSpec (San: Jose, Calif.). Anti-EP2B antibody titers were 120,000 to 125,000 as determined by ELISA using EP2 peptide, not conjugated to KLH, as antigen. Titer was estimated as the dilution at which the optical density was >0.1.

### EXAMPLE 10

Antimicrobial Assays

Strains of Neisseria gonorrhoeae, Chlamydia trachomatis, Pseudomonas aeruginosa and Escherichia coli are obtained from the ATCC and are grown overnight on appropriate agar plates (LB or GCB) at 37° C. ± a 5% CO₂ atmosphere (required for Neisseria gonorrhoeae). The bacteria are removed from the plates inoculated into 5 mls of broth and incubated with shaking at 37° C. to mid-log phase. The cultures are then diluted 1:10,000 with either 0.3% (v/v) LB broth or 20% (v/v) GCB broth.

Candida albicans 16820 is grown with shaking for 24 hours at 37° C. in Sabouraud dextrose broth (SDB). Midlogarithmic phase fungi are obtained by inoculating 1 ml of overnight culture into 50 ml of SDB and incubating 3 h with shaking. The cultures are centrifuged at 10,000 rpm for 10 min at 4° C., washed with cold 10 mM phosphate, pH 7.4, and resuspended in cold buffer.

The antibacterial activity of an EP2 peptide is assayed in 96 well polypropylene microtiter plates. Peptide stock (10 μl 100 μl) is added into a well of the microtiter plate and serially diluted (1:2) through 8 subsequent wells using buffer. The 10th well is the control and receives only 10 μl of buffer. Diluted bacteria (90 μl) are then added into each well and the plate is incubated at 37° C. for 2 h.

Samples (2 μl) from each well are spread onto an agar plate and the agar plates are incubated at 37° C. overnight. Minimal growth inhibitory concentration (MGIC) is the first dilution of EP2 peptide that inhibits all growth on the agar plate. Colony forming units (CFUs) are determined by dilution plating of samples from wells onto the appropriate agar, incubating and counting the colonies formed.

### EXAMPLE 11

Antimicrobial Activity of EP2 Peptides

MGIC for the bacteria Neisseria gonorrhoeae, Chlamydia trachomatis, Pseudomonas aeruginosa and Escherichia coli are determined as in Example 10. For each organism, dilutions of EP2A (SEQ ID NOs:13&14), EP2B (SEQ ID NOs:15&16), EP2C (SEQ ID NOs:17&18), EP2D (SEQ ID NOs:19&20), EP2E (SEQ ID NOs:20&21) and EP2F (SEQ ID NOs:22&23) peptides are made ranging from >500 μg/ml to 1 μg/ml using ¼ strength buffer. Buffer alone is used as a negative control and an appropriate antibiotic is used as appositive control. Bacterial growth is assessed. The EP2 peptides inhibit Neisseria gonorrhoeae, Chlamydia trachomatis, Pseudomonas aeruginosa and Escherichia coli growth.

Fungicidal activity of the EP2 peptides is assessed using 30 μl 10 mM phosphate buffer, 10 μl of Candida albicans stock suspension and 10 μl for a final EP2 peptide concentration of 0.50 μg/ml. The suspensions are incubated for 1 h at 37° C., a 30 μl aliquot is removed, diluted 10, 100 and 1000 fold, and duplicate 100 μl samples of each dilution are spread onto SDB plates and incubated for 18 h at 37° C. Surviving organisms are quantitated. The EP2 peptides inhibit Candida albicans growth.

### EXAMPLE 12

Induction of EP2 Peptide Expression

To enhance native expression of EP2 peptide, an expression vector coding for the EP2 peptide is mnicroinjected into rat epididymis and infused into rat vagina. The expression vector DNA may be mixed with a chemical including, but not limited to, a cationic lipid to enhance the transfection efficiency of the DNA into the epididymal and vaginal cells.

For microinjection, the rat is anaesthetized, a scrotal incision is made and the epididymis is exposed. Using a micromanipulator, a micropipet containing the expression vector in physiological saline is introduced into the caput, corpus, cauda and vas deferens segments of the epididymis and the expression vector is injected. The scrotal incision is closed and the animal is allowed to recover from the surgery.

For infusion, the rat is anaesthetized and the expression vector in physiological saline is infused into the vagina. The animal is allowed to recover from the infusion.

After a suitable period of time, the transfected animals (experimental) and untreated animals (control) are challenged by microinjection (epididymis) or infusion (vagina) with a suspension of Escherichia coli in saline. Experimental animals show a greater resistiance to infection than control animals.

Modifications and variations of the present method will be obvious to those skilled in the art from the foregoing detailed description. Such modifications and variations are intended to come within the scope of the appended claims.

We claim:

1. A composition comprising an isolated nucleic acid having any one of the sequences corresponding to SEQ ID NOs:34 to 44, 49 to 51, 54, 56, 58 to 62, 68, and 69 and degenerate variants of SEQ ID NOs:34 to 44, 49 to 51, 54, 56, 58 to 62, 68 and 69.

2. The composition of claim 1, wherein the isolated nucleic acid has any one of the sequences corresponding to SEQ ID NOs:68 and 69.

3. The composition of claim 1, wherein the isolated nucleic acid has any one of the sequences corresponding to SEQ ID NOs:49 to 51, 54, 56 and 58 to 62.

4. The composition of claim 1, wherein the isolated nucleic acid has any one of the sequences corresponding to SEQ ID NOs:36 to 43.

5. The composition of claim 4, wherein the isolated nucleic acid has any one of the sequences corresponding to SEQ ID NOs:36 to 43 that code for an EP2 defensin.

6. A composition comprising a vector containing an isolated nucleic acid that codes for a peptide having any one of the sequences corresponding to SEQ ID NOs:3 to 12 and 17 to 24 or fragments of SEQ ID NOs:3 to 12 and 17 to 24.

7. The composition of claim 6, wherein the vector is an expression vector.

8. A composition comprising an isolated peptide having any one of the sequences corresponding to SEQ ID NOs:3 to 12, 17 to 24 and 28 to 31 or fragments of SEQ ID NOs:3to 12 and 17 to 24.

9. The composition of claim 8, wherein the isolated peptide has any one of the sequences corresponding to SEQ ID NOs:5 to 12 and 17 to 24.

10. The composition of claim 9, wherein the isolated peptide having any one of the sequences corresponding SEQ ID NOs:17 to 24 is an EP2 defensin.

11. A composition comprising an EP2 peptide, wherein the EP2 peptide has any one of the sequences corresponding to SEQ ID NOs:28 to 31, 25+28, 25+29, 25+30 and 25+31.

12. The composition of claim 11, comprising an EP2 peptide and a pharmaceutically acceptable carrier.

13. A method, wherein a composition comprising an EP2 peptide having any one of the sequences corresponding to SEQ ID NOs:3 to 12, 15 to 24 and 28 to 31 and fragments of SEQ ID NOs:3 to 12, 15 to 24 and 28 to 31 is administered to an animal having an infection in an amount effective to treat the infection in the animal.

14. The method of claim 13, wherein the animal is a primate.

15. The method of claim 14, wherein the primate is a human.

16. The method of claim 13, wherein the infection is a microbial infection.

17. The method of claim 16, wherein the microbial infection is selected from the group consisting of a bacterial, fungal, viral and parasitic infection.

18. The method of claim 13, wherein the infection is an epithelial infection.

19. The method of claim 18, wherein the epithelial infection is a urogenital tract infection.

20. The method of claim 19, wherein the epithelial infection is epididymitis.

21. The method of claim 17, wherein the epithelial infection is a sexually transmitted infection.

22. A method, wherein an anti-EP2 antibody that binds specifically to an EP2 peptide having any one of the sequences corresponding to SEQ ID Nos:3 to 12, 17 to 24 and 28-31 and fragments of SEQ ID Nos:3 to 12, 17 to 24 and 28-31 is used to measure the amount of the EP2 peptide in a body fluid or tissue sample of an animal.

23. A composition comprising an isolated nucleic acid that codes for a peptide comprising at least 25 contiguous residues of a peptide having any one of the sequences corresponding to SEQ ID NOs:28-31 and fragments of SEQ ID NOs:28-31.

24. A composition comprising an isolated nucleic acid, wherein the nucleic acid hybridizes under highly stringent conditions to a nucleic acid having any one of the sequences corresponding to SEQ ID NOs:34 to 44, 49 to 51, 54, 56, 58 to 62, 68, 69 and degenerate variants of SEQ ID NOs:34 to 44, 49 to 51, 54, 56, 58 to 62, 68 and 69.

25. A composition comprising an isolated nucleic acid, wherein the nucleic acid comprises at least 25 consecutive nucleotides of the complement of any one of the sequences corresponding to SEQ ID NOs:34 to 44, 49 to 51, 54, 56, 58 to 62, 68, 69 and degenerate variants of SEQ ID NOs:34 to 44, 49 to 51, 54, 56, 58 to 62, 68 and 69.


SEQUENCE LISTING (69 sequences)

SEQ ID NO: 1 - protein, 103 aa, Homo sapiens [sequence omitted]

SEQ ID NO: 2 - protein, 103 aa, Pan troglodytes
MRQRLLPSVTSLLLVALLFPGSSQARHVNHSATEALGELRERAPGQGTNGFQLLRHAVKRDLLPPRTPPYQVHISHQEARGPSFKICVGFLGPRWARGCSTGN

SEQ ID NO: 3 - protein, 50 aa, Homo sapiens
MKVFFLFAVLFCLVQTNSVHISHQEARGPSFRICVDFLGPRWARGCSTGN

SEQ ID NO: 4 - protein, 50 aa, Pan troglodytes
MKVFFLFAVLFCLVQTNSVHISHQEARGPSFKICVGFLGPRWARGCSTGN

SEQ ID NO: 5 - protein, 113 aa, Homo sapiens
MRQRLLPSVTSLLLVALLFPGSSQARHVNHSATEALGELRERAPGQGTNGFQLLRHAVKRDLLPPRTPPYQEPASDLKVVDCRRSEGFCQEYCNYMETQVGYCSKKKDACCLH

SEQ ID NO: 6 - protein, 113 aa, Pan troglodytes
MRQRLLPSVTSLLLVALLFPGSSQARHVNHSATEALGELRERAPGQGTNGFQLLRHAVKRDLLPPRTPPYQEPASDLKVVDFRRSEGFCQEYCNYMETQVGYCPKKKDACCLH

SEQ ID NO: 7 - protein, 133 aa, Homo sapiens
MRQRLLPSVTSLLLVALLFPGSSQARHVNHSATEALGELRERAPGQGTNGFQLLRHAVKRDLLPPRTPPYQGDVPPGIRNTICRMQQGICRLFFCHSGEKKRDICSDPWNRCCVSNTDEEGKEKPEMDGRSGI

SEQ ID NO: 8 - protein, 133 aa, Pan troglodytes
MRQRLLPSVTSLLLVALLFPGSSQARHVNHSATEALGELRERAPGQGTNGFQLLRHAVKRDLLPPRTPPYQGDVPLGIRNTICRMQQGICRLFFCHSGEKKRDICSDPWNRCCVSNTDEEGKEKPEMDGRSGI

SEQ ID NO: 9 - protein, 80 aa, Homo sapiens
MKVFFLFAVLFCLVQTNSGDVPPGIRNTICRMQQGICRLFFCHSGEKKRDICSDPWNRCCVSNTDEEGKEKPEMDGRSGI

SEQ ID NO: 10 - protein, 80 aa, Pan troglodytes
MKVFFLFAVLFCLVQTNSGDVPLGIRNTICRMQQGICRLFFCHSGEKKRDICSDPWNRCCVSNTDEEGKEKPEMDGRSGI

SEQ ID NO: 11 - protein, 62 aa, Homo sapiens
MRQRLLPSVTSLLLVALLFPEPASDLKVVDCRRSEGFCQEYCNYMETQVGYCSKKKDACCLH

SEQ ID NO: 12 - protein, 62 aa, Pan troglodytes
MRQRLLPSVTSLLLVALLFPEPASDLKVVDFRRSEGFCQEYCNYMETQVGYCPKKKDACCLH

SEQ ID NO: 13 - protein, 79 aa, Homo sapiens
ARHVNHSATEALGELRERAPGQGTNGFQLLRHAVKRDLLPPRTPPYQVHISHQEARGPSFRICVDFLGPRWARGCSTGN

SEQ ID NO: 14 - protein, 79 aa, Pan troglodytes
ARHVNHSATEALGELRERAPGQGTNGFQLLRHAVKRDLLPPRTPPYQVHISHQEARGPSFKICVGFLGPRWARGCSTGN

SEQ ID NO: 15 - protein, 34 aa, Homo sapiens
NSVHISHQEARGPSFRICVDFLGPRWARGCSTGN

SEQ ID NO: 16 - protein, 34 aa, Pan troglodytes
NSVHISHQEARGPSFKICVGFLGPRWARGCSTGN

SEQ ID NO: 17 - protein, 89 aa, Homo sapiens
ARHVNHSATEALGELRERAPGQGTNGFQLLRHAVKRDLLPPRTPPYQEPASDLKVVDCRRSEGFCQEYCNYMETQVGYCSKKKDACCLH

SEQ ID NO: 18 - protein, 89 aa, Pan troglodytes
ARHVNHSATEALGELRERAPGQGTNGFQLLRHAVKRDLLPPRTPPYQEPASDLKVVDFRRSEGFCQEYCNYMETQVGYCPKKKDACCLH

SEQ ID NO: 19 - protein, 109 aa, Homo sapiens
ARHVNHSATEALGELRERAPGQGTNGFQLLRHAVKRDLLPPRTPPYQGDVPPGIRNTICRMQQGICRLFFCHSGEKKRDICSDPWNRCCVSNTDEEGKEKPEMDGRSGI

SEQ ID NO: 20 - protein, 109 aa, Pan troglodytes
ARHVNHSATEALGELRERAPGQGTNGFQLLRHAVKRDLLPPRTPPYQGDVPLGIRNTICRMQQGICRLFFCHSGEKKRDICSDPWNRCCVSNTDEEGKEKPEMDGRSGI

SEQ ID NO: 21 - protein, 64 aa, Homo sapiens
NSGDVPPGIRNTICRMQQGICRLFFCHSGEKKRDICSDPWNRCCVSNTDEEGKEKPEMDGRSGI

SEQ ID NO: 22 - protein, 64 aa, Pan troglodytes
NSGDVPLGIRNTICRMQQGICRLFFCHSGEKKRDICSDPWNRCCVSNTDEEGKEKPEMDGRSGI

SEQ ID NO: 23 - protein, 41 aa, Homo sapiens
PASDLKVVDCRRSEGFCQEYCNYMETQVGYCSKKKDACCLH

SEQ ID NO: 24 - protein, 41 aa, Pan troglodytes
PASDLKVVDFRRSEGFCQEYCNYMETQVGYCPKKKDACCLH

SEQ ID NO: 25 - protein, 47 aa, Homo sapiens
ARHVNHSATEALGELRERAPGQGTNGFQLLRHAVKRDLLPPRTPPYQ

SEQ ID NO: 26 - protein, 32 aa, Homo sapiens
VHISHQEARGPSFRICVDFLGPRWARGCSTGN

SEQ ID NO: 27 - protein, 32 aa, Pan troglodytes
VHISHQEARGPSFKICVGFLGPRWARGCSTGN

SEQ ID NO: 28 - protein, 42 aa, Homo sapiens
EPASDLKVVDCRRSEGFCQEYCNYMETQVGYCSKKKDACCLH

SEQ ID NO: 29 - protein, 42 aa, Pan troglodytes
EPASDLKVVDFRRSEGFCQEYCNYMETQVGYCPKKKDACCLH

SEQ ID NO: 30 - protein, 62 aa, Homo sapiens
GDVPPGIRNTICRMQQGICRLFFCHSGEKKRDICSDPWNRCCVSNTDEEGKEKPEMDGRSGI

SEQ ID NO: 31 - protein, 62 aa, Pan troglodytes
GDVPLGIRNTICRMQQGICRLFFCHSGEKKRDICSDPWNRCCVSNTDEEGKEKPEMDGRSGI

SEQ ID NO: 32 - DNA, 663 bp, Homo sapiens
tgggtgctttctggcttgcagtgctcttggcagacatgaggcaacgattgctcccgtccgtcaccagccttctccttgtggccctgctgtttccaggatcgtctcaagccagacatgtgaaccactcagccactgaggctctcggagaactcagggaaagagcccctgggcaaggcacaaacgggtttcagctgctacgccacgcagtgaaacgggacctcttaccaccgcgcaccccaccttaccaagtgcacatctctcaccaggaggctcgaggaccctcatttaggatctgtgtggactttttagggcctagatgggccaggggatgttccaccgggaattagaaataccatctgccgtatgcagcaagggatctgcagactttttttctgccattctggtgagaaaaagcgtgacatttgctctgatccctggaataggtgttgcgtatcaaatacagatgaagaaggaaaagagaaaccagagatggatggcagatctgggatctaaaatataagctcccggaaggcagggatgttgaagtatcccaagggcttaaaggaatgtgtggcttatagtaggtgttcaataaatatttgttgaatgaatttagcaccaaaggtgaagagctgataaaagacatttttttaacttccttac

SEQ ID NO: 33 - DNA, 647 bp, Pan troglodytes
cttggcagacatgaggcaacgattgctcccgtccgtcaccagccttctccttgtggccctgctgtttccaggatcgtctcaagccagacatgtgaaccactcagccactgaggctctcggagaactcagggaaagagcccctgggcaaggcacaaacgggtttcagctgctacgccacgcagtgaaacgggacctcttaccaccgcgcaccccaccttaccaagtgcacatctctcaccaggaggctcgaggaccctcatttaagatctgcgtgggctttttagggcctagatgggccaggggatgttccactgggaattagaaataccatctgccgtatgcagcaagggatctgcagactttttttctgccattctggtgagaaaaagcgtgacatttgctctgatccctggaataggtgttgcgtatcaaatacagatgaagaaggaaaagagaaaccagagatggatggcagatctgggatctaaaatataagctcccggaaggcagggatgttgaagtatcccaagggcttaaaggaatgtgtggcttatagtaggtgttcaataaatatttgttgaatgaatttagcaccaaaggtgaagagctgataaaagacatttttttaacttccttacaaaaaaaaa

SEQ ID NO: 34 - DNA, 521 bp, Homo sapiens
catatttgtgctccttcacgggagggcagggaggttcaacggaccttaaaacatgaaggtcttttttctgtttgctgttctcttttgtttggtccaaacaaactcagtgcacatctctcaccaggaggctcgaggaccctcatttaggatctgtgtggactttttagggcctagatgggccaggggatgttccaccgggaattagaaataccatctgccgtatgcagcaagggatctgcagactttttttctgccattctggtgagaaaaagcgtgacatttgctctgatccctggaataggtgttgcgtatcaaatacagatgaagaaggaaaagagaaaccagagatggatggcagatctgggatctaaaatataagctcccggaaggcagggatgttgaagtatcccaagggcttaaaggaatgtgtggcttatagtaggtgttcaataaatatttgttgaatgaatttagcaccaaaggtgaagagctgataaaagacatttttttaacttccttac

SEQ ID NO: 35 - DNA, 526 bp, Pan troglodytes
catatttgctctccttcacgggagggcagggaggttcaacggaccttaaaacatgaaggtcttttttctgtttgctgttctcttttgtttggtccaaacaaactcagtgcacatctctcaccaggaggctcgaggaccctcatttaagatctgcgtgggctttttagggcctagatgggccaggggatgttccactgggaattagaaataccatctgccgtatgcagcaagggatctgcagactttttttctgccattctggtgagaaaaagcgtgacatttgctctgatccctggaataggtgttgcgtatcaaatacagatgaagaaggaaaagagaaaccagagatggatggcagatctgggatctaaaatataagctcccggaaggcagggatgttgaagtatcccaagggcttaaaggaatgtgtggcttatagtaggtgttcaataaatatttgttgaatgaatttagcaccaaaggtgaagagctgataaaagagatttttttaacttccttaaaaaaa

SEQ ID NO: 36 - DNA, 397 bp, Homo sapiens
tgggtgctttctggcttgcagtgctcttggcagacatgaggcaacgattgctcccgtccgtcaccagccttctccttgtggccctgctgtttccaggatcgtctcaagccagacatgtgaaccactcagccactgaggctctcggagaactcagggaaagagcccctgggcaaggcacaaacgggtttcagctgctacgccacgcagtgaaacgggacctcttaccaccgcgcaccccaccttaccaagaacctgcatcagatttaaaagttgttgactgcaggagaagtgaaggcttctgccaagaatactgtaattatatggaaacacaagtaggctactgctctaaaaagaaagacgcctgctgtttacattaaaactgatgttgctgatatag

SEQ ID NO: 37 - DNA, 397 bp, Pan troglodytes
ctggcttgcagtgctcttggcagacatgaggcaacgattgctcccgtccgtcaccagccttctccttgtggccctgctgtttccaggatcgtctcaagccagacatgtgaaccactcagccactgaggctctcggagaactcagggaaagagcccctgggcaaggcacaaacgggtttcagctgctacgccacgcagtgaaacgggacctcttaccaccgcgcaccccaccttaccaagaacctgcatcagatttaaaagttgttgacttcaggagaagtgaaggcttctgccaagaatactgtaattatatggaaacacaagtaggctactgccctaaaaagaaagacgcctgctgtttacattaaaactgatgttgctgatatagaaaaaaaaaa

SEQ ID NO: 38 - DNA, 587 bp, Homo sapiens
tgggtgctttctggcttgcagtgctcttggcagacatgaggcaacgattgctcccgtccgtcaccagccttctccttgtggccctgctgtttccaggatcgtctcaagccagacatgtgaaccactcagccactgaggctctcggagaactcagggaaagagcccctgggcaaggcacaaacgggtttcagctgctacgccacgcagtgaaacgggacctcttaccaccgcgcaccccaccttaccaaggggatgttccaccgggaattagaaataccatctgccgtatgcagcaagggatctgcagactttttttctgccattctggtgagaaaaagcgtgacatttgctctgatccctggaataggtgttgcgtatcaaatacagatgaagaaggaaaagagaaaccagagatggatggcagatctgggatctaaaatataagctcccggaaggcagggatgttgaagtatcccaagggcttaaaggaatgtgtggcttatagtaggtgttcaataaatatttgttgaatgaatttagcaccaaaggtgaagagctgataaaagacatttttttaacttccttac

SEQ ID NO: 39 - DNA, 571 bp, Pan troglodytes
cttgcagtgctcttggcagacatgaggcaacgattgctcccgtccgtcaccagccttctccttgtggccctgctgtttccaggatcgtctcaagccagacatgtgaaccactcagccactgaggctctcggagaactcagggaaagagcccctgggcaaggcacaaacgggtttcagctgctacgccacgcagtgaaacgggacctcttaccaccgcgcaccccaccttaccaaggggatgttccactgggaattagaaataccatctgccgtatgcagcaagggatctgcagactttttttctgccattctggtgagaaaaagcgtgacatttgctctgatccctggaataggtgttgcgtatcaaatacagatgaagaaggaaaagagaaaccagagatggatggcagatctgggatctaaaatataagctcccggaaggcagggatgttgaagtatcccaagggcttaaaggaatgtgtggcttatagtaggtgttcaataaatatttgttgaatgaatttagcaccaaaggtgaagagctgataaaagagatttttttaacttcctt

SEQ ID NO: 40 - DNA, 445 bp, Homo sapiens
catatttgtgctccttcacgggagggcagggaggttcaacggaccttaaaacatgaaggtcttttttctgtttgctgttctcttttgtttggtccaaacaaactcaggggatgttccaccgggaattagaaataccatctgccgtatgcagcaagggatctgcagactttttttctgccattctggtgagaaaaagcgtgacatttgctctgatccctggaataggtgttgcgtatcaaatacagatgaagaaggaaaagagaaaccagagatggatggcagatctgggatctaaaatataagctcccggaaggcagggatgttgaagtatcccaagggcttaaaggaatgtgtggcttatagtaggtgttcaataaatatttgttgaatgaatttagcaccaaaggtgaagagctgataaaagacatttttttaacttccttac

SEQ ID NO: 41 - DNA, 443 bp, Pan troglodytes
catatttgctctccttcacgggagggcagggaggttcaacggaccttaaaacatgaaggtcttttttctgtttgctgttctcttttgtttggtccaaacaaactcaggggatgttccactgggaattagaaataccatctgccgtatgcagcaagggatctgcagactttttttctgccattctggtgagaaaaagcgtgacatttgctctgatccctggaataggtgttgcgtatcaaatacagatgaagaaggaaaagagaaaccagagatggatggcagatctgggatctaaaatataagctcccggaaggcagggatgttgaagtatcccaagggcttaaaggaatgtgtggcttatagtaggtgttcaataaatatttgttgaatgaatttagcaccaaaggtgaagagctgataaaagagatttttttaacttcctt

SEQ ID NO: 42 - DNA, 234 bp, Homo sapiens
ctggcttgcagtgctcttggcagacatgaggcaacgattgctcccgtccgtcaccagccttctccttgtggccctgctgtttccagaacctgcatcagatttaaaagttgttgactgcaggagaagtgaaggcttctgccaagaatactgtaattatatggaaacacaagtaggctactgctctaaaaagaaagacgcctgctgtttacattaaaactgatgttgctgatatag

SEQ ID NO: 43 - DNA, 234 bp, Pan troglodytes
ctggcttgcagtgctcttggcagacatgaggcaacgattgctcccgtccgtcaccagccttctccttgtggccctgctgtttccagaacctgcatcagatttaaaagttgttgacttcaggagaagtgaaggcttctgccaagaatactgtaattatatggaaacacaagtaggctactgccctaaaaagaaagacgcctgctgtttacattaaaactgatgttgctgatatag

SEQ ID NO: 44 - DNA, 19426 bp, Homo sapiens
  misc_feature (16627)..(16627): n=any nucleotide
aagaccagcctggccaacatggtgaaaacccgatctctactaaaaatacaaaaattagctgggcgcggtggcaggcacctgtaatcccagctactcaggaggctgaggcaggagagtcgcttgaacccgggaggcggaggttgcagtgagctgagatggcgccgctgcactccagcttgggcaacaatgccagactccgtctcaagaaaaagaataggcaatctcaacagatttatttaaacttataacaataccatgtttttattaccaaaactaaatggtgtttatgccttagcgctcatgaaaggatttcctgtgttctttcatatgctgccttaagagcattcttgggatggctgaaatggctacagatcaaatcgacttctgaaaacacaattcattttgtgattctgtgcatgaaaaagaaacaaaataccaaagaatatttttgcacaattctcaaagctacttctttaaccacgatccaaaagcagttttctctcctatcatgtaattcttcctgactgctttttccaaagaagactctaatatttgtgtcttttccatatatcagttattttccctagaggggaatctgtgcctctgtaaatggcattctagttggtcttacagactggttagcatgttacaatctcagacttaagaataagaaaatctgcataggaatctttgcttcgctcttctgtgagtctcctccagagaaactttcactgggtcatttagtaatgcaaaagaagagtctaaatttgattctgcagagaacttctgattccaaactgggctacaatagggttttccttctcgcattcatattttccaggatttaccaggatgctacttgggaagcaagaaggaggatgtgccgatgtggaaaatcttaccccctgcacatgtgtgcaatttccaataagatccttcaggaatatgtatttgcagaacttcttatttgacaataaaatcttgatcattttactttagcccacctacttagtccaaacgaatcaagataccacatactaagcagcttaaaaaaaaaagaatatgatttattgattgaatggaccaaaaaaaacttgaaacaattattagaatattctataatgggttctgccatcctccccctcaggatggatgtggcttttagcaagagaattattcaaagatttttttaggacacagaaatctggcagaagaggacaggagctgagagcattgttgtgttaggacagatgtaacattaattgcctttattacgacttcaccagcttttgcctgtcaaagagcagaactaggctttcccggctgctcttttttaagattgttcttttcagaagcatggaagagggggcttactttatctcaagacgtagacaaagaaagtgagatctaactatttttggctcagtttcttcatttaaattatttcaaataattctaacgacttaaaagaagattccgttacctgggtgggtaattactcaaaatgctgttatattttaagtcatgattttgattaatgattcattactatgaatatctgaatggtggaataggcttgtttttgttttctttccttttatagagaagataaaaatatatagaaataagttaccaatatactccaaaatttccatcactgttataaaagatccacattccaagtttaaataattacaaatacaactgtaagaagttgctattgaactagagtataaaaaatacccagagtatgtagatgagcgaataaatcttcatttagggttgaggtagagcagctgtctacctcctttcttgactgtctatgttcttccaacatccaattatcagaatttgatgcagtaagtgattaaagaaacttatcatgggccagttgtcacctatctccgcagtgttgccctgtgctcttggaattggaagacttcctaattccttaaagtgaaaggatgtgaatgatgctcctgctctccctgaccagcacctcatgctttgcagtggagaatctgtcctgagacccaaaagatagtggcctccgcattgtgctgccagggcagctgctatgtgcaactgtccgcagctgcaaaccttccgccctttgctggtgcttcagcggatgcccaggtctctgttgtcattgctgcctctttctccatttgcttccagctttctccaggtagagagtaagtatttttatttacacaaatgacctaagttgttttctctgtctggattaaaatatacatgcaaatgagacatatgagataagcactatcttttccagacatcactgatgttacattggatgctatgtgaatacaaaactcttcaaccaaagccttcttcactttagttaagtccagagcagactgtctgggttacatgcatacctgagctaatgcagccaagtaagaaacacacacttggttaaaatgcttaaaaagatgaaggagaagggaagacaagtcctctgcttggatattactagaggagaaaacccagactcaaacacagattttttttttcttttttaaaagaattgaattggacccagtgacatcaacaggaggtgtctgggggtaaagagaatggaaaggggagagaaaaatcaagacaactcaaataagttaaaatagaaaggaggggggtccaaagtgaggaaggagaagtggaggggaccaagaaacagggagagagactcagagaggagaagaaaaagaaaagaacattttgagcagccttggaactctctgtataacttcaggaagggatagtttgtaaaaccaggtcctacctgttatgttgtgtgtcttatgcatgattttttaacactaaaataaaaacgctcagccaacaggatagaatcgacatggcagtttatttatgtccctgttctcatgaacattagggggcttttgagaagcgtttgaggacattggcaactttatgatagttatgtttgttctgcccctccatgcctttcatctttctgtttctctctgttcttccttattcaccaaacccacccaaggcattcaggcgtattatttacttcctgaaatatgtgtctcaagtgtttgttccaccagcagtgggatagtagcgtgtccacattgtcctttgagaatgagaagtcatcctggagcacagctcttcccacgctccgggcccacacacccagcctcactccatcaaaggagccccgctgcctgcccacccaccctgggtgctttctggcttgcagtgctcttggcagacatgaggcaacgattgctcccgtccgtcaccagccttctccttgtggccctgctgtttccaggtaaaatggaaaggtgacccgggtctgggtgccagaatctctctgcaatggtcatctgaggtatgggagtccaggctggacagggagagatgaagtccttggggcatgtattcctggtggagctttgggtacgagtctctgaactgggttcataaatggcactctgaattggctgatggcacttgcttcccagggaagagtgtccctccccgactccattttcttatccttttaacattcccctttcccttacagagaagaacattacattttagggaatcttaacaactgcattagtgacacttgaaataaattctctggctgtgctggctttgaggaggtgctcagactcaccattcatggcatacatttcttacacttcattcaccttctctctctacacataggtgcatacaacgcatgtgcacaaatgtgcacacacacggaacactagcacccctcccaactcccccacccaatcacccatgctcactcacctggtagagtgtaggtgcctcatgctgacgggtctgccaggcggaggcctcagagcatcctcagacgtgtgtttccacttgcacaggatcgtctcaagccagacatgtgaaccactcagccactgaggctctcggagaactcagggaaagagcccctgggcaaggcacaaacgggtttcagctgctacgccacgcagtgaaacgggacctcttaccaccgcgcaccccaccttaccaaggtgagtcagggaccaacacgtgcaacaagtgcatccactggggagacgtagagggaacaaatagacgggaagatgtctgtgctggtcggggtgggtgagcagtcattgtttggggaagacatggtgcgggtgcattgggctgccctgcctgtcagggagaccacggggtctcacagcttcccctggggctggatcattgagggccttgtggaacgtgggagtattgaggggccaaaaggcaatttatctgaagccacacctgtaattgctggctttctccaagaacaggtgccagaggagacactggtggaaacacggcctcctgccaggttgcagccccatgcccttagctttggaggtcgttcccgttcaaggaatttactgaatacctacctactaagtttcaggtgtccattgaggtctgggaaatgcttcctgaggatgggggcagagaatagactgtattgtcagtctactcaggcaaggaggtagcaggacatggcaagggacaattgagcccacagccactcttctggactcttccagaagggccaggcttctggtcagcccccaaaccctgggcaggaccagcttcaaatccaaaagggcctggaagagcctgtagtttccaagatgcttctttaatgccaagctgattgctgaccctaagacagggagaactaggttagcagatcagtgggcaagagcaagaaagacaggagggtgttggcaaattgctgtgacatccagcaaataaagtcctgctgaatttgatgcctgcagcatcctacccaacctcccatccctttctaattggcccacagttccaaaggactcattcatctggatctcctcccaagggaagggaaaaagaaaactcaattattacgcaacaaatataaacaggtgcaatagtaggcgtgtgttatttagtccaacagtattcctgtgagacaggattattccttcctttggttaaaggagatttagtggggcagagctatatatctggagttgatgttttagtttggaacttgaagctattaactatggaaagctgtttcatatccatgccacccccaccctgtccctcacggctcaaatgaccactgtttgattctggagatggtcttaagaagctaatgttggattttttctttttttaatgaagaacctgcatcagatttaaaagttgttgactgcaggagaagtgaaggcttctgccaagaatactgtaattatatggaaacacaagtaggctactgctctaaaaagaaagacgcctgctgtttacattaaaactgatgttgctgatatagaaacaaagctctgccacttacctgttctccggggccacgttgtccaatcaggtgcaggttttttgcggaagtgtctgagcagcagggagcggagatattgccacttgtgccagacagttcaaatattttattgtggcaagataaatgacaaaaatgctacctgtgatcttacagaagatgacttagcttgacatgaacagaattttcaaaatcacacaatttgtgctagtaaatgtggatatcataaacttttattagaaagaattaaaataatttgttcttttatttaaaaactattttttgaatacctacttctattctaggtactgtgcaatggagtccattctattctaagtactgtgaaatgtagttgaggtgtcaggtgtgtggtcaaacccattctccatcagccccatcatctcctacctgcaggccagttcgaagccctgttctctagcagcagctgagagagtgtgcaggcaaatgccttctggggagatactgagagctgggagttactgcctgttctctttgtgctaaacctggaagaataccctctggaagtcctcttgtgcccctttaaaactacccatttgttctctgtggccttagaagacacaaaatgcagagacccatttataacccgatggtgctgtgcaattccaggcttttggtgtcctgaacaaagaactggatgtgatactcagacagcaaagcaagcagcaaaagtgtgtgaagcgcagtattacattcccggagaggggagagtgggctgacttctgccaaatgagattagcatggcttcggtgtaccttgggtctttttatgtgttttttccccttctcttcgcaaggctgcctgatcttttgccggtgcctgccttttgatagataggtgtgttgcttagttactttagcctgtgcgggcttgtgaattgtctccatcccataattttaactacatgcgtgataggtagtccatatgcatgagctttaatgagctgattatcatacagcatcctgttaaggatacttttctctttaatgcgcatgcctatctctgaagagctgcccttcctggtttgatctggatcttgctggccatggggtccttgctctcttctttatctcactttttcttttggctgcttcacttctgccttttatcttgcttcttgctcacccaccccttcaccttgcttctgtttctgcttttattcactctatcctttatccaacttccaatttcctctgcaattctcctgcctcacattaacttctagagataagtgatttaggagccagtcccccaggtggcagctataaaactgtgcttgaagtattctccttcaagggagaagctgragacctggatttcttgctggagctagtcgagaggagcaggcgcagtgcctacctatacctctgttcaggctcccacaggtctactgttcaccctgcccctttggctcccagatacagacccagaagtcaaccctcaggcagcagatgggaaaatgggtagataaaccccttgcaggtagaaaccgggaggtgggcatttacctgcctgctctggcctgagcccagggagagagctgccaaaagtgcttgcaaatctgtgtcccaccatctctttggtgtctgtggtttagggagacctgcagatgcccagctctatcaaccctgagctgggtgatttaggagccaaacccctgggtgggaagcataaaggtcagggtactatatgtgtggtccaaacccttcactcctcagggagaagctaggagttgggacttccttaccaattaattgtaaggtgttatgcctgggatagggattgtgcggggagtgtgtctcagctcttcccacctgttttcacatgaatattttctcagttgcttgatgtgtagtagtcgttcaattagtccatggttttctctcagaaagaaccgatctgtgtgttgatatttctttggtatatccgtggaaggaaggaaaggctggagcctcttagtccaccatcttgcagatatcagtctggcacacccttgattactgtgtagttgagcgttttttcttttgtttattgatcattttttctcttctgtaaaatttaatagttttactgggttactactttattctcttttttcttttttttttgagacagagtttcacccttgttgcccaggctggagtgcaatggtgcgattttggctcaccaccacctccacctcccgggttcaagcgattctcccaggttcaagcaattctcctgcctcagccttcctaagtagctgggattacaggtgtccaccaccatgcctggctaattttgtatttttagtagagacggggtttctccatgttgatcaggctggtctcaaactcccgacctcaggtgatctgtccgcctcggcctcccgaagtgctagaattacaggcgtgagccaccgcgcccggccaggttaccgctttattcttatccatgtacatgtgctccttttatatctaaccttacatattctttcctccatcaattatcttccctttctctggaatgcctcctgcttaaaccccagttatccttgaaatatcctctcattatctttcaaccataattttcatgcttttcccttttgttctctactttgcagatatttatttttatatctgtagagcccttctatttttttttttttttttactatttttggattttgaagttcagttattagatttttaattatttttaaatagtcttatatttgcaaatagtccctttctttctcaacgtgtgactttctcaatgggatctaattctcattttgctgaccacagtttctcaaatctcattaggaataaatacatattgattgttttaaaactgcatttttccattgaaatcgcacatattccagttcagttaagtctcttccagtttctaaaagtgctgacttcacttctgctagttttacactgaccgctaacatctgcttgctcatgcagaccagctgtgcaggattgctttggtagggtaagcaagcgagtgagaggtggcaaagagtgaatgtgggtagaatctgctgttygtcttaaacttttctgtagggtatctctacttaaaagaagtttcatggatgtctttaataaataaatagggaagaaataatgatatttgcctaatctgtccataaaatcttcatcagtgatcattattttaggctcaagttaattaataataaactgcacatcacaaaactttgagctcattattctctttgcagctttctttaatccccataaattaaaacttcctgcaaatattgttatgtattagattgctacaaaagtaattgtggtttttgccattgaaaataacggtcaaaaccgcaattacttttgcaccaacctaaatataagcatctgggtaagaatcaggatcctggggctcagcataggaaagaaactaggtacacagagccctttacagaatgccacctttccaggctttttttctgaacaaagatgttccttaatcaacttatatatgggcacactcacacggggctgatacccacggataatgaaggtatatgaaactagtcctcacatacaaaaccagaagcactggtaacttaaataacctctttgtgaagcaatcttaatatgtatgtaagatataaaatactcgttctcattgtttcagttattctgctcagaaattatcctgggaaataaaaaaatactttgagaaaccaaaaaaaactctaaaaagttcaaaatggagcactatattagcaaactacaataaatctactcagagatattacatattccttaggtataataaatataacacagtgaaaaaccatcaaaatgcataacattttcaaaatacttacatgtcctaaaaattatgataccaatgttcacatatttgatataaaattaactgtgaaaagtatgcttaaaaacaataaagtttctaatgttaaaattattggtgatttttagatatttgcacctttaaaaataccatcaagttattgcattactcttaaatgttaaatatacatttgtaagaacaaaattaatgtgcctaaaaataaacatatatatgtatatacaaagatatcacttaattataagcaaagacattctgttgtagacttaagttgcatccagaaactcacagagttaacataagaaagcgcttcctgggcagtaagatgataagactccttgtttgcatagcggtgagtaaaaataaataaaaattttataaaaagatgatatgacatttaaagcatgttacccagggagactttggagcctacctgggattcataaaaaacaggtagaaactggtttttttttcctctcattctttgatattataatatttgcagtgctgggttgtggacaaggggagggagagcattaggacaaatacttaatgtatgtggggcttagaatatagatgacgggttgatgggtgcagaaaaccaccatggcacatgcatatctatgtaacaaacctgcacgttctgcacatgtatcccagaacttaaagtaaaataaaataaaaataaataaaaattacagacgttaaaaaaacaaatgtgccatcctgcttggagacaactgaataaatataatgggttctcaaggtcagtcctgagccaccaattccgttagttatgcattctttgttctcacagcagctttttagtacaggcccctgagccctgatgaggttttatggccaccagttttacgcagtgggaagaggtccttagacagaagcttgctggaggctgtctcagaacgcactgcagcacacctgactgccttgtattccactctgcacgcccaccttccgcgcagcatccttccctyccctgcaccccarcagcttttcccgggatttgatccttctgactcatccattgctyagagagtccccatcatcaggrarcctgtcttctcttcaatgcctgaggtttgcggggcaaggaacaggtgggcaggctcagtcaattccaccccattgcacctcgtgtgacataaataatgggcgcttctaatcttttcttcctgtccctacatgtggtcgtcaccgcaactctgcaggcttgacctgctctcacctggcttatttttacctctttgggtcatgggaaatgaccttctgcacccagggaatctcccttagttgataagaccaaaatggaaataaataataagaccaaaatggaaagttagtatgccttcataaagagagattaaattcatgaacacaaaccctgcctctttcttgaaaacccaaaatacataaataaataaaacctccggagcaagaggagtaacattagcattgtccatgaggataaaaaagtggggagaaaccccagctgactttttcatcatcccaaaaggagacaccagtaagcagccactggatttgccagctgtgcacatttcatatatatggaatcatacaatatgtggtattttgtgcctagcttattttacttaatgtgagattttcaaagttcctccatgttggagaatgtggcattacttcttttccttttgtgactaaataatattgcattgtatggatgcgccacactttgcttatacattcatcaactgatgcacattygcattgcttccaccgttgacacttgtgactaattctgctatgaacactacactcatgtacaggttgctgtttgaacacctattttcaccgcttgtatgtatacacctaggagttgaattgctaggccatattggtaactcatattgtttaactttctgaggaactgccagacttttccacagmagctgcaccactgyacattcccagcagcaacatatgaaggttacaatgtctccacattctcaccaacacttgctgttttcaaaaattttgttttgttttgtttcattttgagacagagtctcgctctggcgcccaggctggagtgcagtggcccgatctcagctcactgcaacttccgcctcccgggttcaagcgattctcctgcctcagcmtcctgagtagctgggattacaggcacccaccagcatgcctagctaattgcccggctaatttttgtatttttagtagagatggggtttcaccatattggscaggctggtctcgaactcctgaccttgtgatccayccgccttggcctcccaaagtgctgggattacaggtgtgagccactgcttctgaagttttattttttttatgactgtcctagtagatgtgaagtgatatttcattgtggttttgatttgcatgtttctaatgactaatgatattgagcatctttcgtgtgcttgctggtcatttgaatttcttctttggaaaaatctatttaagtcctttgtccatttttaagtgtgttgtttgtctttttgttgttgaattgtatcaatattttttaaaatatagaaacattttttctactatcaaatgtttgcaaacccaaagttatctttcctctcttctccttacactcttcttttccattcatgagtatgatgcacatcagaataattagcttgtgtgttggcacaaattgaactctatttcctttcaactctgcaattatatgaacctatgaacctataaccagatattaacaaaattagccaataagcgtgattttctagtttgatttctttgaaatgatatgccttattcttcagaattatccacaaaatagttccgtggggattgctttctgggtctgtgatttggaaatggattcaagtctggaggagaaggtacatgataaaatttaatactattaatttatttctcccccaaatgaatttattttccaacatagtttattgtttccaaactatacagaaattttctaaactataatttcacaatgatttgattagtaactgtactgctagaaaaaatatgccatccacatttaccttggatcctttccaaataacgtgtagtataaatagaaagaatgaatgtaaagtataaaatatgcattttattgttttatctataagtcatcttagtgacttttaaaaaatgactcaaatttttgaatatccacactcagtgtttttatcaaacaatggttcatgtatcgtacagccactttgtccatgcacaggatacattcagaattgtcattattccttgggacccttgaacttagggtatcatcttggtgtggaagccaatttccctaaggggcaaatgaaattgcttttctttctttctttctttttttttttttttttgagagatttcagagatgtcttcagaacaaatgctccacagagaaagaatttcacattttaatcgatttcttaaagtactgagttggaccctcacaaatattcataactattttacaattacttagtacatagctaacatttaaggtaacttttttttttctctctttttttttggtggagggtcaaaagcagcttggagtgcccaattttccctaaagtcttaacttcaaaggtgattttgcaaggtacagaaaggtctgtgagtcagagagtccctgccagggcacattgtcctgcttaatctctccagaggtggaaagttcaaaatgaacacccagcccctgcctctttgagatgctcacactgttcacccatgcagaaagtccaagaccactgctcgatgtctctttttcaaaatccatgtctaggtaagactcatggtgagatatggttgttgtagactggttaaataacgcagaagacagcttgcagaaaatatgatgtgtctaatctgaagaataacaaggctctgcaagctataacaagtaatataggcaagtccagaatgatatctagagtctgctattgcttacataaaaatggggtatggattcatctctgcttccatacacatgggacacctctggaaggattaataagaagctaacagcaattgttatgaagcccagggggataggagagaagacatccttctcacttgccccttttgcttaagagaattttaagggaaataaatctaagtgatcctgggactaaaatcaaataggggcaaaatgtgcagatttatccactgtgtgttttaataccacacattatataaacccacacacaaaaatatcgtgtccttgcagattctgttttcacaactcccagcaccccagagcccacaaacctccctccagcccaggatgacacagccctgtggttgccgggggctctctgcatccctcactagactgtcacctccgacagcggataacttcatcaaatgagagaagagcatgtcctcttcctccaaagtagacaatatcctggctcttcataaataggtgaattttgccaaatttttggaataatgtggtacgttgtctctgtttttttttttctttcagcatagcgtttgtgaggttcatccacgttgttgtacacatcttcttgtttttctattcttgttgtggtgtggaaatacattgtgttttgttgttgtcataatacactatttggttgcattctcttggctgcaaggaaatgtgctacaatgaacgttcgtgcacatgtctcctaatgcacgtgggcctatgtttctgttggtagatgggagtcatgttgctgggaaataagctaattatctgctcaactgtggtggacaccgcagctttctgaaggcagaaaatatatctttacacaaccatgtctctagcacctagcacagggcttggcactaagtagccacacctcaatgttggttcactttcctcttcaatatccgtatatggaattattggttgatccctgcttctctgaatatcaggaagccagtctatttttaggcagaaagggaagagtagtcagtaaccttctgcccacagccttactcagtagagcagataaatatgctcatgctgatcagtattcccaaaaacctataaatgtcccattttgtgccttctccgctccatttcattccatcattcatcatatttgtgctccttcacgggagggcagggaggttcaacggaccttaaaacatgaaggtcttttttctgtttgctgttctcttttgtttggtccaaacaaactcaggtaaatgtctcctggttagccctggggaaggtagtgcaggaattccatttatgtgtgtgtctgtatggacagtgtgtagatgtgtctgtatgttgttagtggatgcaggtgggccactgtggggctcagtcttggacaattttgatctcccctgtgaagttttttaaaagctaaataagtgttataaaggtcttgacacaagacaaaggggtatgcttgctctgatacaagtggcaagcactcactgcagtctgagaaaagttttcagaaggaagttatagtcatatgaatgtcagagctggaagggaatcagagattgtctatagcagccccatgctctacaaaaagaaaaccaaagtccagagaaagtgttaatttaccatggtgcaatgaatctttatggtcatagtaggtcttcaaacttataatattccccctgcttgcacatagaacacattttacagatgggcaagctgaagtgtaaccagttaaatgagttgtctaaggagacataatgagatattggaagaagtaagaccagaatccaggtctccacgcttccaggctgggggctcttctgtcttgactaaaggtggaccccccaccttcttcactttgctgtctcctccaagctgtgacagggctgagatgatacagaatcagggattagaccccgtttggaggttggatgttgtgcaagagtgttttcctaatcacgcaagaccaacactgtgctgttgttgttgctgctgttgttgctgctgtttaaagtcatcgtacgtagcatttgcagatctgacataagtaagatctttctttcaaccatctcttgcccaatgtcctgttgttataaaaatttaggtggtcatttgtgacttacaagcccacaggtcctggtgaggagagaggttttattttctccttttcgttgtaggacataaaactaaaaattgggccataagttgagaatgggttaatacctctaatttccttagagaccaagacctgtcctattctggaccacttctgttttccaaaactccctttgtttccttctagtgcacatctctcaccaggaggctcgaggaccctcatttaggatctgtgtggactttttagggcctagatgggccaggtgagcattcataaaacacaccctatcatcctcctggcaacatttcagatataaattatcgttcctgttttaaagctaagaggccaaagttcggttaaactggggcttgtccaaaagtacttagccttgtcagaatatataacccttggcagcgggctgggatcatcttctattctctgcactatatgagttaaatgtcaactctcttctgttgtatccataggggatgttccaccgggaattagaaataccatctgccgtatgcagcaagggatctgcagactttttttctgccattctggtgagaaaaagcgtgacatttgctctgatccctggaataggtgttgcgtatcaaatacagatgaagaaggaaaagagaaaccagagatggatggcagatctgggatctaaaatataagctcccggaaggcagggatgttgaagtatcccaagggcttaaaggaatgtgtggcttatagtaggtgttcaataaatatttgttgaatgaatttagcaccaaaggtgaagagctgataaaagacatttttttaacttccttacttctccatgtactgccttttcaaaggggtctcagaattttgtgatattccactttcctttcctagtcaagggaatatctcttaagtatctggagatgggaactgactagaaaccgagctccaaactgattttcagagagacataaatgcaaccaatctgctgctctgktttccttctgatgacatctttcttaccacacccagcactagccttctcctgcttattcacccagatggtaatgcaccttatcccttttccctttatgcctcctcaagcaataacaccaacaggtcacatttcagtaggatagtgttgttttcaagcatttactcttacggctatttcaatttattaatacaacaagctgatagtgtgtataatagggggtaagcaggtccattttaggaatgaaagaanantgaaaatcatcaggtgaagcacatttcccccaggctagcaattcataaatggcattctcagtatgcctatcagtcagcattcattcttctatgatccttctaaaaaacatatttctgtgcaattggagcaaggcagggcccctgttcatggagattcctgaatgattagctgccttttgcctttctctggatcctgcttgaatttgttgaatactaattccaatagtagtgaacaccaattacaagtaaggaatttaaaaaataataaaamcaaacgtgctgagagatagagaccacatgccaagctttttcctgactgacaggtggcttgggaagatgctctgtgttcctgtttctgtcgctgcctgagtccagtgtgcttctgaatgagctgaggtgctgtaaagagcccactagaatgtacactttggggctacagtctcaattcccagctcaagttgcaatgaatatttgtcatcttgcctttggcctctcgcaacctcttctaaactcactcttgttttttttcttttctaaatgcaatcagacagacctctggaagtgcacagagtaagtctctcttaggcacaggcacctctgcagggctctctgtcatgcctctagaggggagagccattgttccatccctgaagggaatgacttcctgaagggcattggcccctattagccttggctcagagtgaaacgcagcaaacgtgcatgcctcagaactggcagacacgttctcagccaggagtgccaccaacccacaccagcagatctgtgtaccagaaacacaaaataacatgaagggccactggggggctggagcctggttccataaggatgggacttctgggcaggtgccgttagacagcagcaccttcttttggcctcatgttcctcagaaatgaaatgaaggaggtgggctcgatctcagactccatcagagcacagcagcctgggggtgtggaagcagagcctcacccaaggaccaaggggtctccaccaggtgagatggggaggatgggaaccccgtccctccctgccagggtgctgcaggtgagaacccccagggagccctctgcagagtccagggccggccagcagggcactcgcgtgggccctaggctgtattatttaatatttttaaggtggaccctgggcctgggctgatctaaattgtggaaaatgtcatctccctcttatgtaaaatttctccaaaggaagtgtttgtccacctgtaaggcatggtaaaaaactagtacctatggcgttgcccagcacaaaacaggctgtctgggagtgtttgctgaggcttccgggaagcaaacatggaagggaagggaagggaaggggaggagaggggaagggaagggacgggagggaaggcggtgcaggctcctggagtcctcagtggtgagctctggagttgctctgttccctttttaatttttgtttactttttggctgttttttctttttctttttttcaatgtaaagtgtctctgtaaggcctgagaatgaatctgactggatcagcccagagaccaagtgagagcccccaaaactggggcttactttcttgttccgcccctctgggatattgggcagatctccatagcatccctgtccccatctgtaaagtgacaggattgaactcagtcctaaaatgtcctggcgtggttctaagacagaatccccaaaacgctctttttcaaagcctgaaaggcttgggtcaggcaggcatcccggcaataccaacacctaccacgcgagggcgcgctgcccttccggcgcctgcagatgggattttttttttttttttttttttttttttttttgaccacttgttctgaagctgggcactgggctaaggacaggagcagctggggtcaccgcaggggagagccagggggcccaggttaccaaagcttctggcctgaatctcttggcactgattacagtgcctcattcgtctctgccctgcacagggtaccattcacgccgggggtgcggaaatgaattaagttcagactgaatcagcagggatatttattgaggcattgtcaggcatctgctcttatttgggaacagggacaggccagcagcacagacaacgctgtggataaggagagtagagacttcctcttcctgcctcctgtcttctggagttgtgaccgcagggtggcccaggtggatcggcttcagaggccaggaggcagctctctgcagccgaagagcaggagcctcacccagggtctgtggctctgactaagcctggactgcctctcggctgtgctccgtggactggctcctcagggatccatgtgagagaccggagtatgatcctcagtgcgaggacaaataaaagtagtgattatgtccaccccatcctgccctccgtccagatctgttttcaacttgaggattcatctgccttgtccttgctaagacaccttcagcctgtggtcaggggaagctgggaagaggtgctgggagacccaggacatcgcaagttgcttctctggctggcactcagaggtgcgtgaaccctctgccaaccctaagaggggcaggagggtgcctggtgatgggccggagctccagccagccagcaggggcagaaggactaggcctggtccaatgggggcccaggatgtttttcttggcaaatcctcatacttttcacatagctctttcttctgagataagtgtgatcatcttccactgtatctctaangaatcagcttcctgagatgacacagtaaccaggaatgacagagctgttccttcc

SEQ ID NO: 45 - DNA, 404 bp, Artificial Sequence
  Synthetic probe
agacatgaggcaacgattgctcccgtccgtcaccagccttctccttgtggccctgctgtttccaggatcgtctcaagccagacatgtgaaccactcagccactgaggctctcggagaactcagggaaagagcccctgggcaaggcacaaacgggtttcagctgctacgccacgcagtgaaacgggacctcttaccaccgcgcaccccaccttaccaagtgcacatctctcaccaggaggctcgaggaccctcatttaagatctgcgtgggctttttagggcctagatgggccaggggatgttccactgggaattagaaataccatctgccgtatgcagcaagggatctgcagactttttttctgccattctggtgagaaaaagcgtgacatttgctctgatccc

SEQ ID NO: 46 - DNA, 22 bp, Artificial Sequence
  reverse primer
gggatcagagcaaatgtcacgc

SEQ ID NO: 47 - DNA, 23 bp, Artificial Sequence
  forward primer
agacatgaggcaacgattgctcc

SEQ ID NO: 48 - DNA, 309 bp, Pan troglodytes
atgaggcaacgattgctcccgtccgtcaccagccttctccttgtggccctgctgtttccaggatcgtctcaagccagacatgtgaaccactcagccactgaggctctcggagaactcagggaaagagcccctgggcaaggcacaaacgggtttcagctgctacgccacgcagtgaaacgggacctcttaccaccgcgcaccccaccttaccaagtgcacatctctcaccaggaggctcgaggaccctcatttaagatctgcgtgggctttttagggcctagatgggccaggggatgttccactgggaat

SEQ ID NO: 49 - DNA, 150 bp, Pan troglodytes
atgaaggtcttttttctgtttgctgttctcttttgtttggtccaaacaaactcagtgcacatctctcaccaggaggctcgaggaccctcatttaagatctgcgtgggctttttagggcctagatgggccaggggatgttccactgggaat

SEQ ID NO: 50 - DNA, 339 bp, Pan troglodytes
atgaggcaacgattgctcccgtccgtcaccagccttctccttgtggccctgctgtttccaggatcgtctcaagccagacatgtgaaccactcagccactgaggctctcggagaactcagggaaagagcccctgggcaaggcacaaacgggtttcagctgctacgccacgcagtgaaacgggacctcttaccaccgcgcaccccaccttaccaagaacctgcatcagatttaaaagttgttgacttcaggagaagtgaaggcttctgccaagaatactgtaattatatggaaacacaagtaggctactgccctaaaaagaaagacgcctgctgtttacat

SEQ ID NO: 51 - DNA, 399 bp, Pan troglodytes
atgaggcaacgattgctcccgtccgtcaccagccttctccttgtggccctgctgtttccaggatcgtctcaagccagacatgtgaaccactcagccactgaggctctcggagaactcagggaaagagcccctgggcaaggcacaaacgggtttcagctgctacgccacgcagtgaaacgggacctcttaccaccgcgcaccccaccttaccaaggggatgttccactgggaattagaaataccatctgccgtatgcagcaagggatctgcagactttttttctgccattctggtgagaaaaagcgtgacatttgctctgatccctggaataggtgttgcgtatcaaatacagatgaagaaggaaaagagaaaccagagatggatggcagatctgggatc

SEQ ID NO: 52 - DNA, 20 bp, Artificial Sequence
  reverse primer
cccttgggatacttcaacat

SEQ ID NO: 53 - DNA, 20 bp, Artificial Sequence
  reverse primer
ggcagggaggttcaacggac

SEQ ID NO: 54 - DNA, 240 bp, Pan troglodytes
atgaaggtcttttttctgtttgctgttctcttttgtttggtccaaacaaactcaggggatgttccactgggaattagaaataccatctgccgtatgcagcaagggatctgcagactttttttctgccattctggtgagaaaaagcgtgacatttgctctgatccctggaataggtgttgcgtatcaaatacagatgaagaaggaaaagagaaaccagagatggatggcagatctgggatc

SEQ ID NO: 55 - DNA, 19 bp, Artificial Sequence
  reverse primer
ggcagggaggttcaacgga

SEQ ID NO: 56 - DNA, 186 bp, Pan troglodytes
atgaggcaacgattgctcccgtccgtcaccagccttctccttgtggccctgctgtttccagaacctgcatcagatttaaaagttgttgacttcaggagaagtgaaggcttctgccaagaatactgtaattatatggaaacacaagtaggctactgccctaaaaagaaagacgcctgctgtttacat

SEQ ID NO: 57 - DNA, 309 bp, Homo sapiens
atgaggcaacgattgctcccgtccgtcaccagccttctccttgtggccctgctgtttccaggatcgtctcaagccagacatgtgaaccactcagccactgaggctctcggagaactcagggaaagagcccctgggcaaggcacaaacgggtttcagctgctacgccacgcagtgaaacgggacctcttaccaccgcgcaccccaccttaccaagtgcacatctctcaccaggaggctcgaggaccctcatttaggatctgtgtggactttttagggcctagatgggccaggggatgttccaccgggaat

SEQ ID NO: 58 - DNA, 150 bp, Homo sapiens
atgaaggtcttttttctgtttgctgttctcttttgtttggtccaaacaaactcagtgcacatctctcaccaggaggctcgaggaccctcatttaggatctgtgtggactttttagggcctagatgggccaggggatgttccaccgggaat

SEQ ID NO: 59 - DNA, 339 bp, Homo sapiens
atgaggcaacgattgctcccgtccgtcaccagccttctccttgtggccctgctgtttccaggatcgtctcaagccagacatgtgaaccactcagccactgaggctctcggagaactcagggaaagagcccctgggcaaggcacaaacgggtttcagctgctacgccacgcagtgaaacgggacctcttaccaccgcgcaccccaccttaccaagaacctgcatcagatttaaaagttgttgactgcaggagaagtgaaggcttctgccaagaatactgtaattatatggaaacacaagtaggctactgctctaaaaagaaagacgcctgctgtttacat

SEQ ID NO: 60 - DNA, 399 bp, Homo sapiens
atgaggcaacgattgctcccgtccgtcaccagccttctccttgtggccctgctgtttccaggatcgtctcaagccagacatgtgaaccactcagccactgaggctctcggagaactcagggaaagagcccctgggcaaggcacaaacgggtttcagctgctacgccacgcagtgaaacgggacctcttaccaccgcgcaccccaccttaccaaggggatgttccaccgggaattagaaataccatctgccgtatgcagcaagggatctgcagactttttttctgccattctggtgagaaaaagcgtgacatttgctctgatccctggaataggtgttgcgtatcaaatacagatgaagaaggaaaagagaaaccagagatggatggcagatctgggatc

SEQ ID NO: 61 - DNA, 240 bp, Homo sapiens
atgaaggtcttttttctgtttgctgttctcttttgtttggtccaaacaaactcaggggatgttccaccgggaattagaaataccatctgccgtatgcagcaagggatctgcagactttttttctgccattctggtgagaaaaagcgtgacatttgctctgatccctggaataggtgttgcgtatcaaatacagatgaagaaggaaaagagaaaccagagatggatggcagatctgggatc

SEQ ID NO: 62 - DNA, 186 bp, Homo sapiens
atgaggcaacgattgctcccgtccgtcaccagccttctccttgtggccctgctgtttccagaacctgcatcagatttaaaagttgttgactgcaggagaagtgaaggcttctgccaagaatactgtaattatatggaaacacaagtaggctactgctctaaaaagaaagacgcctgctgtttacat

SEQ ID NO: 63 - protein, 47 aa, Homo sapiens
GNFLTGLGHRSDHYNCVSSGGQCLYSACPIFTKIQGTCYRGKAKCCK

SEQ ID NO: 64 - protein, 41 aa, Homo sapiens
GIGDPVTCLKSGAICHPVFCPRRYKQIGTCGLPGTKCCKKP

SEQ ID NO: 65 - DNA, 20 bp, Artificial Sequence
  forward primer
gacatttgctctgatccctg

SEQ ID NO: 66 - DNA, 24 bp, Artificial Sequence
  forward primer
ggccccagtcactcaggagagatc

SEQ ID NO: 67 - DNA, 21 bp, Artificial Sequence
  reverse primer
cgcatcagccacagcagcttc

SEQ ID NO: 68 - DNA, 3000 bp, Homo sapiens
aatggtgtttatgccttagcgctcatgaaaggatttcctgtgttctttcatatgctgccttaagagcattcttgggatggctgaaatggctacagatcaaatcgacttctgaaaacacaattcattttgtgattctgtgcatgaaaaagaaacaaaataccaaagaatatttttgcacaattctcaaagctacttctttaaccacgatccaaaagcagttttctctcctatcatgtaattcttcctgactgctttttccaaagaagactctaatatttgtgtcttttccatatatcagttattttccctagaggggaatctgtgcctctgtaaatggcattctagttggtcttacagactggttagcatgttacaatctcagacttaagaataagaaaatctgcataggaatctttgcttcgctcttctgtgagtctcctccagagaaactttcactgggtcatttagtaatgcaaaagaagagtctaaatttgattctgcagagaacttctgattccaaactgggctacaatagggttttccttctcgcattcatattttccaggatttaccaggatgctacttgggaagcaagaaggaggatgtgccgatgtggaaaatcttaccccctgcacatgtgtgcaatttccaataagatccttcaggaatatgtatttgcagaacttcttatttgacaataaaatcttgatcattttactttagcccacctacttagtccaaacgaatcaagataccacatactaagcagcttaaaaaaaaaagaatatgatttattgattgaatggaccaaaaaaaacttgaaacaattattagaatattctataatgggttctgccatcctccccctcaggatggatgtggcttttagcaagagaattattcaaagatttttttaggacacagaaatctggcagaagaggacaggagctgagagcattgttgtgttaggacagatgtaacattaattgcctttattacgacttcaccagcttttgcctgtcaaagagcagaactaggctttcccggctgctcttttttaagattgttcttttcagaagcatggaagagggggcttactttatctcaagacgtagacaaagaaagtgagatctaactatttttggctcagtttcttcatttaaattatttcaaataattctaacgacttaaaagaagattccgttacctgggtgggtaattactcaaaatgctgttatattttaagtcatgattttgattaatgattcattactatgaatatctgaatggtggaataggcttgtttttgttttctttccttttatagagaagataaaaatatatagaaataagttaccaatatactccaaaatttccatcactgttataaaagatccacattccaagtttaaataattacaaatacaactgtaagaagttgctattgaactagagtataaaaaatacccagagtatgtagatgagcgaataaatcttcatttagggttgaggtagagcagctgtctacctcctttcttgactgtctatgttcttccaacatccaattatcagaatttgatgcagtaagtgattaaagaaacttatcatgggccagttgtcacctatctccgcagtgttgccctgtgctcttggaattggaagacttcctaattccttaaagtgaaaggatgtgaatgatgctcctgctctccctgaccagcacctcatgctttgcagtggagaatctgtcctgagacccaaaagatagtggcctccgcattgtgctgccagggcagctgctatgtgcaactgtccgcagctgcaaaccttccgccctttgctggtgcttcagcggatgcccaggtctctgttgtcattgctgcctctttctccatttgcttccagctttctccaggtagagagtaagtatttttatttacacaaatgacctaagttgttttctctgtctggattaaaatatacatgcaaatgagacatatgagataagcactatcttttccagacatcactgatgttacattggatgctatgtgaatacaaaactcttcaaccaaagccttcttcactttagttaagtccagagcagactgtctgggttacatgcatacctgagctaatgcagccaagtaagaaacacacacttggttaaaatgcttaaaaagatgaaggagaagggaagacaagtcctctgcttggatattactagaggagaaaacccagactcaaacacagattttttttttcttttttaaaagaattgaattggacccagtgacatcaacaggaggtgtctgggggtaaagagaatggaaaggggagagaaaaatcaagacaactcaaataagttaaaatagaaaggaggggggtccaaagtgaggaaggagaagtggaggggaccaagaaacagggagagagactcagagaggagaagaaaaagaaaagaacattttgagcagccttggaactctctgtataacttcaggaagggatagtttgtaaaaccaggtcctacctgttatgttgtgtgtcttatgcatgattttttaacactaaaataaaaacgctcagccaacaggatagaatcgacatggcagtttatttatgtccctgttctcatgaacattagggggcttttgagaagcgtttgaggacattggcaactttatgatagttatgtttgttctgcccctccatgcctttcatctttctgtttctctctgttcttccttattcaccaaacccacccaaggcattcaggcgtattatttacttcctgaaatatgtgtctcaagtgtttgttccaccagcagtgggatagtagcgtgtccacattgtcctttgagaatgagaagtcatcctggagcacagctcttcccacgctccgggcccacacacccagcctcactccatcaaaggagccccgctgcctgcccacccaccctgggtgctttctggcttgcagtgctcttggcagacatg

SEQ ID NO: 69 - DNA, 3000 bp, Homo sapiens
agtagctgggattacaggcacccaccagcatgcctagctaattgcccggctaatttttgtatttttagtagagatggggtttcaccatattggscaggctggtctcgaactcctgaccttgtgatccayccgccttggcctcccaaagtgctgggattacaggtgtgagccactgcttctgaagttttattttttttatgactgtcctagtagatgtgaagtgatatttcattgtggttttgatttgcatgtttctaatgactaatgatattgagcatctttcgtgtgcttgctggtcatttgaatttcttctttggaaaaatctatttaagtcctttgtccatttttaagtgtgttgtttgtctttttgttgttgaattgtatcaatattttttaaaatatagaaacattttttctactatcaaatgtttgcaaacccaaagttatctttcctctcttctccttacactcttcttttccattcatgagtatgatgcacatcagaataattagcttgtgtgttggcacaaattgaactctatttcctttcaactctgcaattatatgaacctatgaacctataaccagatattaacaaaattagccaataagcgtgattttctagtttgatttctttgaaatgatatgccttattcttcagaattatccacaaaatagttccgtggggattgctttctgggtctgtgatttggaaatggattcaagtctggaggagaaggtacatgataaaatttaatactattaatttatttctcccccaaatgaatttattttccaacatagtttattgtttccaaactatacagaaattttctaaactataatttcacaatgatttgattagtaactgtactgctagaaaaaatatgccatccacatttaccttggatcctttccaaataacgtgtagtataaatagaaagaatgaatgtaaagtataaaatatgcattttattgttttatctataagtcatcttagtgacttttaaaaaatgactcaaatttttgaatatccacactcagtgtttttatcaaacaatggttcatgtatcgtacagccactttgtccatgcacaggatacattcagaattgtcattattccttgggacccttgaacttagggtatcatcttggtgtggaagccaatttccctaaggggcaaatgaaattgcttttctttctttctttctttttttttttttttttgagagatttcagagatgtcttcagaacaaatgctccacagagaaagaatttcacattttaatcgatttcttaaagtactgagttggaccctcacaaatattcataactattttacaattacttagtacatagctaacatttaaggtaacttttttttttctctctttttttttggtggagggtcaaaagcagcttggagtgcccaattttccctaaagtcttaacttcaaaggtgattttgcaaggtacagaaaggtctgtgagtcagagagtccctgccagggcacattgtcctgcttaatctctccagaggtggaaagttcaaaatgaacacccagcccctgcctctttgagatgctcacactgttcacccatgcagaaagtccaagaccactgctcgatgtctctttttcaaaatccatgtctaggtaagactcatggtgagatatggttgttgtagactggttaaataacgcagaagacagcttgcagaaaatatgatgtgtctaatctgaagaataacaaggctctgcaagctataacaagtaatataggcaagtccagaatgatatctagagtctgctattgcttacataaaaatggggtatggattcatctctgcttccatacacatgggacacctctggaaggattaataagaagctaacagcaattgttatgaagcccagggggataggagagaagacatccttctcacttgccccttttgcttaagagaattttaagggaaataaatctaagtgatcctgggactaaaatcaaataggggcaaaatgtgcagatttatccactgtgtgttttaataccacacattatataaacccacacacaaaaatatcgtgtccttgcagattctgttttcacaactcccagcaccccagagcccacaaacctccctccagcccaggatgacacagccctgtggttgccgggggctctctgcatccctcactagactgtcacctccgacagcggataacttcatcaaatgagagaagagcatgtcctcttcctccaaagtagacaatatcctggctcttcataaataggtgaattttgccaaatttttggaataatgtggtacgttgtctctgtttttttttttctttcagcatagcgtttgtgaggttcatccacgttgttgtacacatcttcttgtttttctattcttgttgtggtgtggaaatacattgtgttttgttgttgtcataatacactatttggttgcattctcttggctgcaaggaaatgtgctacaatgaacgttcgtgcacatgtctcctaatgcacgtgggcctatgtttctgttggtagatgggagtcatgttgctgggaaataagctaattatctgctcaactgtggtggacaccgcagctttctgaaggcagaaaatatatctttacacaaccatgtctctagcacctagcacagggcttggcactaagtagccacacctcaatgttggttcactttcctcttcaatatccgtatatggaattattggttgatccctgcttctctgaatatcaggaagccagtctatttttaggcagaaagggaagagtagtcagtaaccttctgcccacagccttactcagtagagcagataaatatgctcatgctgatcagtattcccaaaaacctataaatgtcccattttgtgccttctccgctccatttcattccatcattcatcatatttgtgctccttcacgggagggcagggaggttcaacggaccttaaaacatg